SEQUENCE LISTING (62 sequences)

SEQ ID NO: 1 - protein, 26 aa, Artificial Sequence
  Synthetic peptide
LHHLLHHLLHHLHHLLHHLHHLLHHL

SEQ ID NO: 2 - protein, 26 aa, Artificial Sequence
  Synthetic peptide
LHKLLHHLLHKLHHLLHKLHHLLHHL

SEQ ID NO: 3 - protein, 26 aa, Artificial Sequence
  Synthetic peptide
LHKLLHHLLHHLHKLLHHLHHLLHKL

SEQ ID NO: 4 - protein, 15 aa, Artificial Sequence
  Synthetic peptide
LHHLLHHLLHHLHHL

SEQ ID NO: 5 - protein, 14 aa, Artificial Sequence
  Synthetic peptide
HHLLHHLHHLLHHL

SEQ ID NO: 6 - protein, 14 aa, Artificial Sequence
  Synthetic peptide
LHLLHHLLHHLHHL

SEQ ID NO: 7 - protein, 15 aa, Artificial Sequence
  Synthetic peptide
LHHLLHLLHHLLHHL

SEQ ID NO: 8 - protein, 14 aa, Artificial Sequence
  Synthetic peptide
LHKLLHHLLHHLHK

SEQ ID NO: 9 - protein, 15 aa, Artificial Sequence
  Synthetic peptide
LHKLLHHLHHLLHKL

SEQ ID NO: 10 - protein, 15 aa, Artificial Sequence
  Synthetic peptide
KLHHLLHKLHHLLHH

SEQ ID NO: 11 - protein, 11 aa, Artificial Sequence
  Synthetic peptide
HLHLLHHLLHH

SEQ ID NO: 12 - protein, 10 aa, Artificial Sequence
  Synthetic peptide
LHLLHHLLHH

SEQ ID NO: 13 - protein, 15 aa, Artificial Sequence
  Synthetic peptide
LHKLLHHLLHKLHHL

SEQ ID NO: 14 - protein, 6 aa, Artificial Sequence
  Synthetic peptide
LHLLHH

SEQ ID NO: 15 - protein, 5 aa, Artificial Sequence
  Synthetic peptide
LHHLL

SEQ ID NO: 16 - protein, 5 aa, Artificial Sequence
  Synthetic peptide
LHKLL

SEQ ID NO: 17 - protein, 2 aa, Artificial Sequence
  Synthetic peptide
HH

SEQ ID NO: 18 - protein, 6 aa, Artificial Sequence
  Synthetic peptide
HHHHHH

SEQ ID NO: 19 - protein, 8 aa, Artificial Sequence
  Synthetic peptide
HHHHHHHH

SEQ ID NO: 20 - protein, 15 aa, Artificial Sequence
  Synthetic peptide
HHHHHHHHHHHHHHH

SEQ ID NO: 21 - protein, 2 aa, Artificial Sequence
  Synthetic peptide
LL

SEQ ID NO: 22 - protein, 5 aa, Artificial Sequence
  Synthetic peptide
LLLLL

SEQ ID NO: 23 - protein, 10 aa, Artificial Sequence
  Synthetic peptide
LLLLLLLLLL

SEQ ID NO: 24 - protein, 15 aa, Artificial Sequence
  Synthetic peptide
LLLLLLLLLLLLLLL

SEQ ID NO: 25 - protein, 16 aa, Artificial Sequence
  Synthetic peptide, Penetratin
RQIKIWFQNRRMKWKK

SEQ ID NO: 26 - protein, 27 aa, Artificial Sequence
  Synthetic peptide, Transportan
LIKKALAALAKLNIKGLLYGASNLTWG

SEQ ID NO: 27 - protein, 23 aa, Artificial Sequence
  Synthetic peptide, EB1
LIRLWSHLIHIWFQNRRLKWKKK

SEQ ID NO: 28 - protein, 13 aa, Artificial Sequence
  Synthetic peptide, TAT
GRKKRRQRRRPPQ

SEQ ID NO: 29 - protein, 27 aa, Artificial Sequence
  Synthetic peptide, MPG
GALFLGFLGAAGSTMGAWSQPKKKRKV

SEQ ID NO: 30 - protein, 20 aa, Artificial Sequence
  Synthetic peptide, CADY
GLWRALWRLLRSLWRLLWRA

SEQ ID NO: 31 - protein, 18 aa, Artificial Sequence
  Synthetic peptide, MAP
KLALKLALKALKAALKLA

SEQ ID NO: 32 - protein, 9 aa, Artificial Sequence
  Synthetic peptide, Polyarginine
RRRRRRRRR

SEQ ID NO: 33 - protein, 30 aa, Artificial Sequence
  Synthetic peptide, bPrPp
MVKSKIGSWILVLFVAMWSDVGLCKKRPKP

SEQ ID NO: 34 - protein, 26 aa, Artificial Sequence
  Synthetic peptide
LHHLLHHLLHHLHHLLHHLHHLLHHL

SEQ ID NO: 35 - protein, 26 aa, Artificial Sequence
  Synthetic peptide
LHKLLHHLLHHLHKLLHHLHHLLHKL

SEQ ID NO: 36 - protein, 26 aa, Artificial Sequence
  Synthetic peptide
LHKLLHHLLHKLHHLLHKLHHLLHHL

SEQ ID NO: 37 - protein, 15 aa, Artificial Sequence
  Synthetic peptide
LHHLLHHLLHHLHHL

SEQ ID NO: 38 - protein, 14 aa, Artificial Sequence
  Synthetic peptide
HHLLHHLHHLLHHL

SEQ ID NO: 39 - protein, 14 aa, Artificial Sequence
  Synthetic peptide, sDP(14)
LHLLHHLLHHLHHL

SEQ ID NO: 40 - protein, 15 aa, Artificial Sequence
  Synthetic peptide
LHHLLHLLHHLLHHL

SEQ ID NO: 41 - protein, 14 aa, Artificial Sequence
  Synthetic peptide
LHKLLHHLLHHLHK

SEQ ID NO: 42 - protein, 15 aa, Artificial Sequence
  Synthetic peptide
LHKLLHHLHHLLHKL

SEQ ID NO: 43 - protein, 15 aa, Artificial Sequence
  Synthetic peptide
KLHHLLHKLHHLLHH

SEQ ID NO: 44 - protein, 11 aa, Artificial Sequence
  Synthetic peptide
HLHLLHHLLHH

SEQ ID NO: 45 - protein, 5 aa, Artificial Sequence
  Synthetic peptide, sDP(10)
LHKLL

SEQ ID NO: 46 - protein, 15 aa, Artificial Sequence
  Synthetic peptide
LHKLLHHLLHKLHHL

SEQ ID NO: 47 - protein, 6 aa, Artificial Sequence
  Synthetic peptide
LHLLHH

SEQ ID NO: 48 - protein, 5 aa, Artificial Sequence
  Synthetic peptide, sDP(5)
LHHLL

SEQ ID NO: 49 - protein, 5 aa, Artificial Sequence
  Synthetic peptide
LHKLL

SEQ ID NO: 50 - RNA, 19 nt, Artificial Sequence
  Synthetic siRNA, Target Scr
cagucgcguuugcgacugg

SEQ ID NO: 51 - RNA, 19 nt, Artificial Sequence
  Synthetic siRNA, Target F4/80
gugaaugagugucaagaua

SEQ ID NO: 52 - RNA, 19 nt, Artificial Sequence
  Synthetic siRNA, Target OPN
ccacauggcuggugccuga

SEQ ID NO: 53 - RNA, 19 nt, Artificial Sequence
  Synthetic RNA
gugaaugagugucaagaua

SEQ ID NO: 54 - protein, 2 aa, Artificial Sequence
  Synthetic peptide
HH

SEQ ID NO: 55 - protein, 6 aa, Artificial Sequence
  Synthetic peptide
HHHHHH

SEQ ID NO: 56 - protein, 8 aa, Artificial Sequence
  Synthetic peptide
HHHHHHHH

SEQ ID NO: 57 - protein, 15 aa, Artificial Sequence
  Synthetic peptide
HHHHHHHHHHHHHHH

SEQ ID NO: 58 - protein, 2 aa, Artificial Sequence
  Synthetic peptide
LL

SEQ ID NO: 59 - protein, 5 aa, Artificial Sequence
  Synthetic peptide
LLLLL

SEQ ID NO: 60 - protein, 10 aa, Artificial Sequence
  Synthetic peptide
LLLLLLLLLL

SEQ ID NO: 61 - protein, 15 aa, Artificial Sequence
  Synthetic peptide
LLLLLLLLLLLLLLL

SEQ ID NO: 62 - protein, 26 aa, Artificial Sequence
  Synthetic peptide
LHHLLHHLLHHLHHLLHHLHHLLHHL

(19) United States
(12) Patent Application Publication
(10) Pub. No.: US 2016/0184449 A1
(43) Pub. Date: Jun. 30, 2016

(54) PEPTIDE-AND AMINE-MODIFIED GLUCAN PARTICLES FOR DELIVERY OF THERAPEUTIC CARGOES

(71) Applicant: University of Massachusetts, Boston, MA (US)

(72) Inventors: Michael P. Czech, Westborough, MA (US); Jessica L. Cohen, Worcester, MA (US); Myriam Aouadi, Worcester, MA (US); Yuefei Shen, Boston, MA (US)

(21) Appl. No.: 14/763,308

(22) PCT Filed: Feb. 28, 2014
(86) PCT No.: PCT/US2014/019547

§ 371 Date: Jul. 24, 2015

Related U.S. Application Data
(60) Provisional application No. 61/770,899, filed on Feb. 28, 2013.

(51) Int. Cl. A61K 47/48; C12N 15/113; A61K 36/064; A61K 38/14; A61K 48/00

(52) U.S. Cl. CPC A61K 47/48315; A61K 38/14; A61K 48/0033; A61K 36/064; C12N 15/113; C12N 15/1136; C12N 2310/14; C12N 2310/3513; C12N 2320/30

(57) ABSTRACT

The present invention generally relates to peptide-modified glucan particles (PcGPs) and/or amine-modifies glucan particles (amGPs) for use in delivering payload molecules, in particular, nucleic acid payload molecules, to cells. The invention relates to particular peptides and small molecule amines which are ideally suited for use in the pcGPs and amGPs of the invention, respectively. Preferred aspects of the invention feature a single-component delivery vehicle comprising the pcGPs or amGPs and siRNA, for use as an siRNA delivery vehicle. Methods of making such particles and methods of using such particles, for example, for in mediating in vitro and in vivo gene silencing are disclosed herein.

### GOVERNMENT SUPPORT

This invention was made with government support under National Institutes of Health Grant No. DK085753 and National Institutes of Health/National Institute of Diabetes and Digestive and Kidney Diseases Grant No. NIH NIDDK-F32DK098879. The government has certain rights in the invention.

### BACKGROUND OF THE INVENTION

Gene therapy has enormous potential for the treatment or prevention of various diseases, including cancer, viral infections, and a number of inherited and acquired diseases. RNA interference (RNAi), in particular, has emerged as a promising therapeutic strategy for the treatment of diseases that may not otherwise be accessed with current therapeutic technology. RNA interference (RNAi) is a biological mechanism wherein double-stranded RNAs can be used to reduce expression of target proteins, and has emerged as a promising therapeutic strategy for the treatment of many diseases. Synthetic short interfering RNA (siRNA) can activate the endogenous RNAi pathway to achieve highly efficient, sequence-specific gene silencing. The discovery that synthetic small interfering RNA (siRNA) could achieve sequence-specific gene knockdown in mammalian cells by exploiting the RNAi machinery has lead to a surge of research using RNAi in therapeutic applications. Since synthetic siRNA can be designed to target nearly any gene in the body, therapeutic strategies based on this technology offer advantages over small molecule drugs. siRNA-based therapeutics are currently being developed for a variety of disease indications, including ocular and retinal disorders, cancer, and viral infections.

Despite great therapeutic potential, the clinical application of siRNA is limited by delivery problems. Multiple barriers impede the successful delivery of siRNA to specific tissues in vivo. siRNA does not cross cellular membranes efficiently (i.e., does not readily cross membranes to enter cells) due to its relatively large size, negative charge, and hydrophilicity. In addition, siRNA is unstable under in vivo conditions due to rapid degradation by serum nucleases, i.e., siRNA is rapidly degraded by nucleases and quickly cleared by the kidneys and liver. In addition, inefficient release of siRNA from intracellular membranes into the cytoplasm limits the ability of siRNA to be incorporated into the RNAi machinery and to induce gene silencing. Thus, the widespread use of RNAi therapeutics for disease prevention and treatment requires the development of clinically suitable, safe, and effective delivery vehicles.

Both viral and non-viral carriers have been developed for the delivery of siRNA. Viral-based vectors have been widely studied for the delivery of genetic material due to their relatively high transfection efficiency compared to non-viral vectors. Although viral vectors are very efficient, they can cause immunogenic and inflammatory responses, which raise concerns about their safety as delivery vectors.

This has led to increasing research efforts focused on the design of efficient non-viral delivery systems. Non-viral vectors provide opportunities for improved safety, greater flexibility, and more facile manufacturing. The most common non-viral vectors involve complexes formed between cationic lipids or polymers and siRNA through electrostatic interactions. However, most of these systems suffer from low transfection efficiencies and high toxicity due to their polycationic nature.

Significant progress has been made in the delivery of siRNA therapeutics as evidenced by a number of clinical trials being performed using siRNA or other small RNA mimics. Despite these advances, several challenges to the clinical translation of siRNA remain and, thus, motivate the investigation of alternative siRNA delivery systems that may be able to overcome these obstacles.

### SUMMARY OF THE INVENTION

Phagocytic cells, such as macrophages, are particularly attractive targets for RNA interference therapy because they are causally associated with the development of diseases such as rheumatoid arthritis, atherosclerosis, and diabetes. Macrophages play a critical role in the pathogenesis of these and other inflammatory diseases. In the diseased state, macrophages secrete pro-inflammatory cytokines such as interleukin-1 (IL-1), interleukin-6 (IL-6), tumor necrosis factor-α (TNF-α), and osteopontin (OPN), which induce pathogenic inflammatory responses. The key role that macrophages play in the mechanism of these diseases has lead to therapeutic strategies focused on reducing inflammation by targeting chemokines and cytokines. Thus, siRNA delivery systems capable of targeting phagocytic cells present a promising therapeutic approach for the treatment of these and numerous other major human diseases.

The present invention features siRNA delivery systems capable of targeting phagocytic cells, such as macrophages, as well as therapeutic methods featuring such delivery systems. In one aspect, the invention relates to the preparation and utilization of peptide-modified (e.g., peptide-conjugated) glucan particles for the delivery of therapeutic cargoes. Peptides of varying length and composition are chemically conjugated to glucan particles extracted from yeast cell walls. In another aspect, the invention relates to amine-modified (e.g., conjugated) glucan particles, in particular, small molecule amine-modified glucan particles for the delivery of therapeutic cargoes. Amine-modified glucan particles of the invention comprise small molecule amines, e.g., primary amines, secondary amines, tertiary amines conjugates to the particles, i.e., to glucan particles or glucan shells. Without being bound in theory, it is believed that the amine functionality is particularly amenable to therapeutic targeting of gut inflammatory cytokines. As will be appreciated by the skilled artisan, the conjugated and/or modified particles of the invention are particularly suited for oral administration, and are capable of delivering therapeutic cargoes to the gut or digestive system of a subject (for example, to the colon and/or small intestine.)

It has been discovered by the instant inventors that both the size and charges of conjugated and/or modified particles of the invention affect their delivery to a specific site, e.g., to the gut (for example, to the colon and/or small intestine.) Amine-modified glucan particles show improved delivery. Small molecule amine-modified glucan particles are preferred. It has also been discovered by the instant inventors that adding amine functionality to certain peptides enhances conjugation of same to glucan particles and, perhaps, enhances delivery of peptide-conjugated glucan particles comprising same.

These peptide-modified glucan particles and amine-modified glucan particles are capable of electrostatically binding to negatively-charged cargo (e.g., genetic material). Covalent attachment of peptides (e.g., amphipathic peptides) and/or small molecule amines to β-1,3-D-glucan particles facilitated electrostatic interaction of particles with negatively-charged cargo (e.g., genetic material), in particular, with siRNA. Importantly, they are a single component delivery system. In studies described herein, peptide-modified glucan particles (i.e., peptide-conjugated glucan particles) were used to deliver siRNA targeting F4/80 to peritoneal macrophages in mice. Using obesity as a model of inflammation, i.p. administration of peptide-conjugated glucan particles, loaded with a siRNA targeting the inflammatory cytokine osteopontin (opn), reduced the expression of opn in the adipose tissue macrophages of obese insulin-resistant mice. Using an animal model for colitis, oral administration of amine-modified glucan particles, loaded with an siRNA targeting tumor necrosis alpha (tnf-α), reduced the expression of tnf-α in gut, without affecting circulating levels of certain cytokines. Reduction of colitis-associated weight loss was also demonstrated.

These studies have demonstrated that the new particles can efficiently deliver siRNA in vivo with minimal toxicity. Due to the flexibility of the system, peptide-modified glucan particles (e.g., peptide-conjugated glucan particles) and/or amine-modified glucan particles can potentially be used to deliver siRNA targeting any protein of interest, and thus can be used in the treatment of a number of diseases. Therefore, peptide-modified glucan particles and/or amine-modified glucan particles represent a promising vehicle for the delivery of therapeutic cargoes, specifically gene-silencing siRNA.

The present invention generally relates to peptide-conjugated glucan particles (pcGPs) and/or amine-modified glucan particles (amGPs) useful for receptor-targeted drug delivery. In particular, the present invention relates to pcGPs and/or amGPs containing substances (e.g., therapeutic substances) loaded within such pcGPs or amGPs for use as in vivo delivery systems. The present invention further relates to methods of making a pcGPs and/or amGPs, for example, pcGPs or amGPs loaded with therapeutic substances. The present invention also relates to methods of using the loaded pcGPs and/or amGPs, for example, the use of pcGPs or amGPs, loaded with therapeutic substances, in particular, siRNA payloads, for receptor-targeted delivery of said substances.

In a preferred aspect, the pcGPs and/or amGPS are useful for treating inflammatory diseases, for example, rheumatoid arthritis, psoriasis, asthma, atherosclerosis, and diabetes. In preferred aspects, the pcGPs and/or amGPs are useful for inflammatory diseases of the gut, for example, inflammatory bowel diseases (e.g., Crohn's disease, colitis, for example, ulcerative colitis, and the like.)

The foregoing description broadly describes the features and technical advantages of certain embodiments of the present invention. Further technical advantages will be described in the detailed description of the invention that follows. Novel features that are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to be definitions of the invention's scope.

### BRIEF DESCRIPTION OF DRAWINGS

FIG. 1. Peptide-conjugated GPs (pcGPs) and amine-modified GPs (amGPs) are a simplified single-component system for siRNA delivery. Schematic diagrams of loading strategies for (a) GeRPs and (b) new, peptide-conjugated glucan particles and/or amine-modified glucan particles (GPs). (a) The loading strategy for art-described formulation of GeRPs involves (1) formation of siRNA/delivery peptide (DP) complexes, (2) siRNA/DP complex loading in a hydrodynamic volume into the glucan shells, and (3) entrapment of the complexes in the particles. (b) Peptide-conjugated glucan particles and/or amine-modified glucan particles can be loaded with siRNA simply by mixing siRNA with glucan particles pre-conjugated with peptides and/or small molecule amines, e.g., short delivery peptides (sDPs, for example EP-derived peptides). Covalent attachment of peptides and/or small molecule amines to the glucan shell facilitates not only electrostatic binding of siRNA to this single component delivery system but also escape from the endosomes. (c) General method for the covalent coupling of peptide(s) and/or amines to glucan particles via reductive amination chemistry

FIG. 2. Peptide-conjugated GPs bind to siRNA. Peptide-conjugated GPs (10 mg/mL in acetate buffer) were incubated with different concentrations of fluorescently-labeled siRNA to form electrostatic complexes. Particles loaded with siRNA were sedimented by centrifugation and the supernatant was assessed for siRNA content. (a) Percentage of bound siRNA was plotted against siRNA loading (nmol of siRNA/mg of particles) to determine the binding affinity of the particles. Particles with higher binding affinity have a higher percentage of bound siRNA at increasing siRNA loadings. (b) Amount of siRNA bound to the particles was plotted against siRNA concentration (μM).

FIG. 3. pcGP-14s can silence genes in macrophages in vivo in healthy, lean mice. (a) Time line of pcGP-14 administration and isolation of peritoneal exudate cells (PECs). Briefly, 8-week old C57B16/J mice were injected once a day for 5 days with pcGP-14s loaded with either Scr (black) or F4/80 (stippled) siRNA. On day 6, mice were sacrificed and PECs were isolated. (b) FACS analysis showing PECs isolated from mice treated with Cascade Blue-labeled GeRP-14s and stained with a CD11b-PerCp-Cy5.5 antibody. (c) Peptide-conjugated glucan shells attenuate F4/80 mRNA expression in PECs. Mice were treated with sDP(14)-GS loaded with Scr (black) or F4/80 (stippled) siRNA and F4/80 mRNA expression was measured by real-time PCR in PECs. n=9, statistical significance was determined by student's t-test. **p<0.01. Results are mean±s.e.m.

FIG. 4. Peptide-conjugated glucan shells undergo phagocytosis by macrophages in the epididymal adipose tissue of obese mice. FACS analysis showing adipose tissue macrophages containing peptide-conjugated particles. 5-week old ob/ob mice were injected once a day for 5 days with PBS or 1 mg of fluorescently-labeled sDP(14)-GS loaded with 10 nmoles of Scr siRNA. On day 7, mice were sacrificed and stromal vascular fraction (SVF) cells from the epididymal adipose tissue were isolated, stained, and analyzed by flow cytometry. The macrophage population was defined as F4/80⁺/CD11b⁺/Gr-1⁻. Figure depicts FACS analysis showing that epididiymal adipose tissue macrophages phagocytose peptide-conjugated particles

FIG. 5. Peptide-conjugated glucan shells can reduce and/or attenuate inflammatory cytokines in adipose tissue of obese mice. (a) Time line of peptide-modified glucan shell administration and adipose tissue isolation. Briefly, 5-week old ob/ob mice were injected once a day for 5 days with 1 mg of sDP(14)-GS (pcGP-14) loaded with 10 nmoles of Scr (black) or OPN (stippled) siRNA. On day 7, mice were sacrificed and epididymal adipose tissue (AT) was isolated. Total RNA was extracted and (b) OPN, (c) F4/80, and (d) TNF-α gene expression was measured by real-time PCR in epididymal AT from treated mice. n=15, (e) Glucose tolerance tests were performed on mice that were fasted for 16 h. 5-week old ob/ob mice were injected once a day for 5 days with PBS (cross-hatch) or 1 mg sDP(14)-GS loaded with 10 nmoles of Scr (black) or OPN (stippled) siRNA. On day 6, mice were fasted overnight. On day 7, glucose tolerance tests (GTT) were performed. Briefly, blood glucose was measured before and 15, 30, 60, and 90 minutes after i.p. injecting 1 g/kg of glucose. (f) Area under the curve (A.U.C) was calculated using GraphPad Prism, n=10, statistical significance was determined by student's t-test. ****p<0.0001. Results are mean±s.e.m.

FIG. 6. sDP(14)-GS mediated gene silencing is specific to the epididymal adipose tissue. Figure depicts expression of OPN in (a) liver and (b) subcutaneous (SQ) adipose tissue from mice treated for 5 days with sDP(14)-GS (pcGP-14) loaded with Scr (black) or OPN (cross-hatch) siRNA. n=10, statistical significance was determined by student's t-test. Results are mean±s.e.m.

FIG. 7. In vivo toxicity of pcGP-14s in WT mice. Mice were injected for 5 days with PBS (cross-hatch) or pcGP-14s loaded with Scr (black) or F4/80 (stippled) siRNA. Serum was collected the day following the last injection. (a) Serum AST and (b) ALT activity levels were measured following the 5-day treatment. (c) Serum levels of IFN-γ were measured using ELISA assay.

FIG. 8. Examples of pcGP and amGP formulations binding siRNA. pcGPs or amGPs (1 mg/ml) were incubated with different concentrations of fluorescent siRNAs to form electrostatic complexes in sodium acetate buffer (pH=4.8). After spinning down at 5000 rpm for 5 min, siRNA/GP complexes were precipitated, while free siRNAs were remained in solution. Percentage of free siRNA was then measured and plotted against siRNA concentration to determine the binding affinity of particles.

FIG. 9. (a) Silencing of TNF-α expression in distal colons and (b) Reducing body weight loss of DSS-treated mice, by oral treatment of ethylenediamine-modified amGP formulation in DSS-induced colitis mouse model. C57BL6 male mice treated with water or water containing 3% DSS for 7 days, were given amGPs with control scrambled (SCR) siRNA or with siRNA targeting TNF-α by oral gavage on days 2, 4, and 6. Weight loss was monitored during the treatment. On day 7, different parts of the colon were harvested and RNA was prepared by RNeasy Plus Mini Kit (Qiagen). TNF-α mRNA level was determined by quantitative RT-PCR relative to the mRNA levels of the reference gene 36B4 (**p≦0.01, ***p≦0.001, ****p≦0.0001) The exemplary TNF-α sequence is as follows: 5′-GUGAAUGAGUGUCAAGAUA-3′ (SEQ ID NO: ______).

### DETAILED DESCRIPTION OF THE INVENTION

Peptide-conjugated glucan particles (pcGPs) and amine-modified glucan particles (amGPs) represent promising new particulate delivery systems that improve upon prior art in therapeutic delivery systems. Previous glucan particle (GP)-based delivery technologies had been described in the art. Such delivery technologies, when used to deliver nucleic acid cargoes or payloads, e.g., small interfering RNA (siRNA), generally featured two different approaches. The first approach utilizes a complex, multi-component, layer-by-layer synthesis approach to encapsulate payload (see e.g., Aouadi et al., Nature. 2009; 458(7242): 1180-1184). Components include, for example, glucan particles, siRNA, cationic trapping polymer, e.g., polyethyleneimine (PEI), and core anionic material, e.g., tRNA.

An alternative approach utilizes fewer components for encapsulating the desired therapeutic payload, e.g, an siRNA. Components include glucan shells loaded with complexes of siRNA and, for example, an amphipathic delivery peptide known as Endoporter™, which facilitates transport of siRNA into cells (see e.g., Tesz et al. Biochem. J. (2011) 436, 351-362.) Endoporter (EP) is an amphipathic α-helical peptide, with one face composed predominantly of aliphatic lipophilic amino acids, and the other face composed of basic amino acids. In the context of the glucan shells loaded with complexes of siRNA, EP serves multiple functions in the overall process of siRNA delivery, including entrapping the siRNA within the glucan shells and aiding in its endosomal escape.

The present invention features simplified “one-component” delivery vehicles, which consist essentially of a chemically-modified glucan shell with covalently conjugated short peptides or small molecule amines that can readily encapsulate the therapeutic siRNA payload. Thus while prior art (see, for example, Nature. 2009; 458(7242): 1180-118 or Biochem, J. (2011) 436, 351-362) with glucan particles utilizes multiple, separate components, the system of the present invention is a single (“one-component”) entity. In addition, the prior art formulations, for example, those containing the Endoporter™ peptide, showed immunogenic potential due to the length of the peptide used. The present inventors discovered that covalent attachment of EP, or preferably, shorter peptides containing weak-base residues, to the glucan shell would facilitate siRNA delivery while significantly simplifying the delivery technology. Since the formulations presented herein feature peptides that are chemically conjugated to the glucan particles, the new delivery system is a single entity that can efficiently deliver siRNA while using a short (non-immunogenic) peptide sequence that is now covalently attached to the glucan shell.

The present inventors also discovered that adding amine functionality to GPs significantly enhances the therapeutic potential of modified glucan particles. In particular, amine functionality can be added to GPs by conjugation of small molecule amines to said GPs. Such amine-modified GPs (amGPs) are particularly suited to therapeutic applications where delivery of siRNA cargo to acidic environments is contemplated.

Accordingly, the present invention features, in exemplary aspects, preparations of peptide-conjugated (pc) yeast cell wall particles (YCWPs) (also referred to herein as peptide-conjugated (pc) glucan particles (GPs)), such preparations including peptide, e.g., delivery peptide, conjugated to components, e.g., oligosaccharides, within the cell wall of the YCWPs (GPs). In preferred embodiments, the invention features peptide-conjugated (pc) yeast cell wall particle (YCWP) delivery systems (e.g., peptide-conjugated (pc) glucan particles (GPs), which include YCWPs (GPs) including select yeast cell wall components, e.g., oligosaccharides, conjugated to peptides, e.g., delivery peptides, as delivery systems. Preferably, the systems are for delivery of nucleic acid molecules, e.g., siRNA molecules. Accordingly, preferred aspect of the invention feature the delivery systems wherein nucleic acid payload molecules (e.g., siRNAs) are complexed with peptides, e.g., delivery peptides, within the delivery systems conjugated onto YCWP (GP) walls or within YCWP (GP) walls. In exemplary embodiments, the peptides, e.g., delivery peptides, are selected from the group consisting of cationic peptides, amphipathic peptides, and polyhistidine peptides. Other exemplary peptides are described infra. Delivery peptides can be short in nature, e.g., can have a length of about 5 to about 30 residues. Exemplary amphipathic peptides can have alternating lipophillic (e.g., leucine (L)) and basic (e.g., histidine (H) or lysine (K)) residues, optionally in pairs, optionally alternating with single lipophillic or basic residues. Exemplary amphipathic peptides can have about 50% lipophillic and 50% basic or weakly basic residues, about 40% lipophillic and 60% basic residues, about 60% lipophillic and 40% basic residues, and/or can have the formula [(A)ₙ₌₁₋₂ (B)ₙ₌₁₋₂]ₙ₌₅₋₃₀.

In other exemplary aspects, the invention features polyhistidine peptides for conjugating payload molecules, e.g., nucleic acid molecules, to YCWPs (GPs), for example, the peptides having 2-20, 2-16, 2-10, 2-8 or 2-6 histidines. In other exemplary aspects, the invention features polyleucine peptides for conjugating payload molecules, e.g., nucleic acid molecules, to GPs, for example, the peptides having 2-20, 2-16, 2-10, 2-8 or 2-6 leucines. In other exemplary aspects, the invention features cationic peptides for conjugating payload molecules, e.g., nucleic acid molecules, to YCWPs (GPs), for example, polyarginine peptides, cell-penetrating peptides or other synthetic cationic peptides.

The amino acid sequences of exemplary peptides for use in the invention are set forth infra. Various truncated forms of said peptides are also contemplated for use in the invention.

The present invention further features, in exemplary aspects, preparations of amine-modified (am) glucan particles (GPs) (also referred to herein as amine-modified (am) yeast cell wall particles (YCWPs)), such preparations including small molecule amines conjugated to components, e.g., oligosaccharides, within the cell wall of the YCWPs (GPs). In preferred embodiments, the invention features amine-modified (am) glucan particle (GP) delivery systems, which include (GPs) including select yeast cell wall components, e.g., oligosaccharides, conjugated to small molecule amines, as delivery systems. Preferably, the systems are for delivery of nucleic acid molecules, e.g., siRNA molecules. Accordingly, preferred aspects of the invention feature the delivery systems wherein a nucleic acid payload molecules (e.g., siRNAs) are complexed with small molecule amines, within the delivery systems conjugated onto GP walls or within GP walls.

In exemplary embodiments, the preparations or systems of the invention feature the above-described peptides or small molecule amines conjugated to a moiety in the yeast cell wall particle via a linker moiety. Exemplary linker moieties are a non-degradable linker moieties, or degradable linker moieties, as described in detail infra.

In exemplary aspects, the invention features pcGPs and/or amGPs which include a nucleic acid payload, e.g., siRNA. Accordingly, featured in the invention are methods of effecting gene silencing, e.g., RNAi, of a target gene or mRNA of a target gene in a cell, where the methods include the steps of contacting the cell with an effective amount of a preparation or delivery system of the invention, and incubating the cell under conditions such that the target gene is effectively silenced. Such contacting can be performed in vitro or in vivo. In alternative aspects, the inventions features methods of effecting gene silencing, e.g., RNAi, of a target gene or mRNA of a target gene in an organism, where the methods include the steps of administering to the organism an effective amount of the preparation or delivery system of the invention, under conditions such that the target gene is effectively silenced. In exemplary embodiments, the organism is a subject, e.g., a human subject. In preferred aspects, the methods are for therapeutic purposes. Accordingly, subjects (e.g., human subjects) can have, or be at risk for, developing a disease or disorder associated with the presence of the target gene or mRNA of such a gene.

Further featured aspects of the invention include kits that include one or more preparations or delivery systems of the invention in combination with, at least, specific instructions for use. Also featured are methods of making the preparations or systems of the invention. Such methods can include, for example, the steps of contacting a preparation of YCWPs (GPs) with a peptide (e.g., delivery peptide), where the peptide is modified with a linker moiety, or with a small molecule amine, where the amine is modified with a linker moiety, under conditions such that the peptide is conjugated to a component of the YCWP (GP) via said linker moiety. Alternatively, such methods can include the steps of contacting a peptide-conjugated (pc) GP or amine-modified (am) GP with a nucleic acid payload molecule (e.g., siRNA) under conditions such that the nucleic acid payload molecule complexes with the peptide or small molecule amine within the GP (e.g., within the wall of the GP).

Prior to describing the invention, it may be helpful to an understanding thereof to set forth definitions of certain terms to be used hereinafter.

### I. Definitions

As used herein, the term “peptide-conjugated glucan particle” (“pcGP”) refers to a yeast cell wall particle (YCWP) having conjugated, i.e., chemically conjugated, to it a peptide which imparts a desired functionality to said “peptide-conjugated glucan particle” (“pcGP”). In preferred aspects of the invention, the peptide facilitates delivery of substances capable of complexing with or loading into or onto the pcGPs into the cytosol of cells via an endocytosis-mediated process when said particles are contacted with cells, for example, mammalian cells. In exemplary aspects, such peptides are referred to herein as “delivery peptides” (“DPs”). In certain preferred aspects, such peptides are short in nature, e.g., 2-20 residues in length, referred to herein as “short delivery peptides” (“sDPs”), described in detail infra. Exemplary peptides are amphipathic in nature. The terms “peptide-modified glucan particle” (“pmGP”) or “peptide-modified glucan shell” (“pmGS”) can be used interchangeably herein with the term “peptide-conjugated glucan particle” (“pcGP”) or “peptide-conjugated glucan shell” (“pcGS”).

As used herein, the term “amine-modifed glucan particle” (“amGP”) refers to a yeast cell wall particle having conjugated, i.e., chemically conjugated, to it a small molecule amine which imparts a desired functionality to said “amine modified glucan particle” (“amGP”). In preferred aspects of the invention, the small molecule amine facilitates delivery of substances capable of complexing with or loading into or onto the amGPs into the cytosol of cells via an endocytosis-mediated process when said particles are contacted with cells, for example, mammalian cells.

As used herein, the term “small molecule” refers to a low molecular weight (<900 Daltons) organic compound that is involved or regulates one or more biological processes.

As used herein, the term “small molecule amine” refers to a low molecular weight (<900 Daltons) organic compound comprising, or preferably consisting of carbon, nitrogen, hydrogen and oxygen, that is involved or regulates one or more biological processes. In certain preferred aspects, such “small molecule amines” are small in nature, e.g., 20-100 Dalton (Da), 40-100 Da, 50-100 Da, 50-200 Da, 100-200 Da, or 100-500 Da. Exemplary “small molecule amines” include aliphatic chains containing primary, secondary and/or tertiary amines, e.g., straight or branched chains, or alicyclic chains, saturated or unsaturated chains, etc., and also include chains comprised primarily of carbon but optionally comprising one or more other elements, for example, oxygen, nitrogen, sulfur, chloride, and the like. Exemplary “small molecule amines” also include aromatic chains containing primary, secondary and/or tertiary amines, e.g., aromatic ring-containing chains (aryl amines), heteroaromatic ring containing chains (heteroaryl amines) (e.g., one or more ring atoms selected from elements such as oxygen, sulfur, nitrogen, etc.) and the like. The term “amine-modified glucan particle” (“amGP”) can be used interchangeably herein with the term “amine-modified glucan particle” (“amGP”).

As used herein, the term amphipathic refers to a compound, e.g, a peptide possessing both hydrophilic (“water-loving”, polar) and lipophilic (“fat-loving”) properties. Such a compound is also referred to in the art as an “amphiphilic” compound e.g., peptide.

As used herein, the term “yeast cell wall particle” (“YCWP”) refers to a micron-sized β-glucan shell or particle resulting from alkaline, acid and/or solvent extraction of yeast to remove cytoplasmic components and/or other proteins and/or polysaccharides. An exemplary “yeast cell wall particle” (“YCWP”) is derived from a naturally-occurring yeast cell, having at least one component of the yeast cell wall modified, e.g., extracted. An exemplary yeast cell is Saccharomyces cerevisiae. S. cerevisiae cell walls comprise components including, but not limited to, glucan, chitosan, mannan, proteins and the like. Extraction of one or more of these components from a naturally occurring yeast cell wall results in a YCWP. Based on their size, YCWPs can also be referred to herein and in the art as microparticles.

As used herein, the term yeast glucan mannan particle (“YGMP”) or glucan mannan particle (“GMP”) or glucan mannan shell (“GMS”) refers to a YCWP comprising a significant percentage of yeast cell wall β-glucan and mannan. The extraction process is such that a significant percentage of the β-glucan and mannan present in the source yeast cell wall remains in the particle following extraction.

As used herein, the term yeast glucan particle (“YGP”) or glucan particle (“GP”), or glucan shell (“GS”) refers to a YCWP comprising primarily yeast cell wall β-glucan. Harsher extraction conditions are used as compared to those for making YGMPs such that mannan is removed from the particle walls.

As used herein, the term yeast chitosan/mannan particle (“YCMP”) or chitosan/mannan particle (“CMP”) or chitosan/mannan shell (“CMS”) refers to a YCWP comprising a significant percentage of yeast cell wall chitosan and mannan.

The extraction process is such that a significant percentage of the chitosan and mannan present in the source yeast cell wall remains in the particle following extraction.

As used herein, the term “glucan encapsulated siRNA particle” (“GeRP”) refers to a YCWP, GP or glucan shell containing siRNA encapsulated within.

As used herein, the term “nanoparticle” refers to a particle (e.g., a spherical particle) of less than 1 micron in diameter, e.g., 500, 200, 100, 50 nanometers or less in diameter.

As used herein, the term “microparticle” refers to a particle (e.g., a spherical particle) of greater than (or equal to) 1 micron in diameter, e.g., 1, 10, 50, 100 and up to 999 micrometers in diameter.

As used herein, the term “payload molecule” or “payload agent” refers to an agent or molecule (e.g., a pharmaceutically active agent) of interest which is delivered or released from pcGPs of the invention or a delivery system comprising said pcGPs. In exemplary embodiments, a payload molecule is selected from the group consisting of a nucleic acid, a peptide, a protein, a small organic active agent, a small inorganic active agent, and a mixture thereof. Preferred payload molecules are nucleic acid molecules, e.g., siRNA molecules. For example, the payload molecule may be a therapeutic agent or a diagnostic nucleic acid molecule, e.g., siRNA molecule.

As used herein, the term “load” refers to the introduction or insertion or complexing of a substance (e.g., a payload or payload molecule) into or onto a particle of the invention, for example, a pcGP or amGP. As used herein, the term “loading” refers to combining or incubating the substance to be delivered (e.g., a payload or payload molecule) with the particles (e.g., pcGPs or amGPs) of the invention under conditions and/or for a time period sufficient for the substance to sufficiently incorporate therein. In exemplary embodiments, the substance to be loaded into, or loaded into or complexed with, particles of the invention (e.g., pcGPs or amGPs) are anionic payloads, in particular, siRNAs.

As used herein, the term “delivery system” refers to a combination of reagents (and, optionally, procedures) used to achieve delivery of a desired payload or cargo into a cell or into a desired compartment within a cell.

“Essentially pure” means a composition comprising at least about 90% by weight of the desired molecule, based on total weight of the composition, preferably at least about 95% by weight. “Essentially homogeneous” means a composition comprising at least about 99% by weight of the desired molecule, based on total weight of the composition.

The term “drug” refers to a substance that, when absorbed into the body of a living organism, alters normal bodily function.

The term “patient” includes human and other mammalian subjects that receive either prophylactic or therapeutic treatment.

The term “effective dose” or “effective dosage” is defined as an amount sufficient to achieve or at least partially achieve the desired effect. The term “therapeutically effective dose” is defined as an amount sufficient to cure or at least partially arrest a disease and/or its complications in a patient already suffering from the disease.

The invention features pcGPs and/or amGPs loaded with payload molecules e.g., loaded with payload molecules having desired functionalities. The following sections provide detailed descriptions of methods of making and using exemplary pcGPs amGPs of the invention.

### II. Methods of Making Yeast Cell Wall Particles (YCWPs)

YCWPs, e.g., YGPs or GPs, YGMPs or GMPs, etc., exhibit key functional properties of the native or naturally-occurring yeast from which they are derived. Extracted yeast cell wall particles, primarily due to their beta-glucan content, are targeted to phagocytic cells, such as macrophages and cells of lymphoid tissue. The mucosal-associated lymphoid tissue (MAT) comprises all lymphoid cells in epithelia and in the lamina propria lying below the body's mucosal surfaces. The main sites of mucosal-associated lymphoid tissues are the gut-associated lymphoid tissues (GALT), the bronchial-associated lymphoid tissues (BALT), and the SALT skin-associated lymphoid tissue (SALT).

Another important component of the GI immune system is the M or microfold cell. M cells are a specific cell type in the intestinal epithelium over lymphoid follicles that endocytose a variety of protein and peptide antigens. Instead of digesting these proteins, M cells transport them into the underlying tissue, where they are taken up by local dendritic cells and macrophages. M cells are another target of YCWPs on the invention. M cells take up molecules and particles from the gut lumen by endocytosis or phagocytosis. This material is then transported through the interior of the cell in vesicles to the basal cell membrane, where it is released into the extracellular space. This process is known as transcytosis and has been shown to occur effectively for uptake of YCWPs (see e.g., Beier and Gebert, Am J. Physiol. 1998 July; 275 (1 Pt 1):G130-7 and van der Lubben, et al., J Drug Target, 2002 September; 10 (6):449-56). Without being bound in theory, it is believed that uptake of YCWPs into cells, for example macrophage cells, occurs at least in part due to specific receptor-mediated uptake. For example, it is known that pathogen pattern recognition receptors (PRRs) recognize common structural and molecular motifs present on microbial surfaces and that mannose receptors and beta-glucan receptors, in part, participate in the recognition of fungal pathogens.

The mannose receptor (MR), a carbohydrate-binding receptor expressed on subsets of macrophages, is considered one such PRR. Macrophages have receptors for both mannose and mannose-6-phosphate that can bind to and internalize molecules displaying these sugars. The molecules are internalized by endocytosis into a pre-lysosomal phagosome. This internalization has been used to enhance entry of oligonucleotides into macrophages using bovine serum albumin modified with mannose-6-phosphate and linked to an oligodeoxynucleotide by a disulfide bridge to a modified 3′ end. See, e.g., Bonfils, et al., Nucl. Acids Res. 1992 20, 4621-4629; and Bonfils, et al., Bioconj. Chem., 3, 277-284 (1992).

Macrophages also express beta-glucan receptors, including CR3 (Ross, et al., 1987, Complement Inflamm. 4:61), dectin-1. (Brown and Gordon. 2001. Nature 413:36.), and lactosylceramide (Zimmerman et al., J Biol. Chem. 1998 Aug. 21:273 (34):22014-20.). The beta-glucan receptor, CR₃ is predominantly expressed on monocytes, neutrophils and NK cells, whereas dectin-1 is predominantly expressed on the surface of cells of the macrophages. Lactosylceramide is found at high levels in M cells. Microglia can also express a beta-glucan receptor (Muller, et al., Res Immunol. 1994 May; 145 (4):267-75). There is evidence for additive effects on phagocytosis of binding to both mannose and beta-glucan receptors. (Giaimis, J., et al., J Leukoc Biol. 1993 December; 54 (6):564-71).

In some embodiments, hollow beta 1,3-D-glucan microsphere-based delivery system incorporating payloads as polymer complexes can be used. The payload molecules can include for example, plasmid/mRNA molecules, protein molecules, nanoparticles, siRNA/oligonucleotides, up to 200% w/w small molecules. In certain embodiments, a multiplexed co-delivery of different payloads is possible. In various embodiments, beta glucan receptor-mediated uptake by antigen presenting cells can be performed. The methods of the invention can be utilized for delivering payload(s) to dendritic cells, macrophages and tissues containing these cells.

Exemplary extracted yeast cell wall particles (YCWPs) are readily available, biodegradable, substantially spherical particles about 2-4 um in diameter. Preparation of extracted yeast cell wall particles is described infra.

A. Yeast Glucan Particles and Yeast Glucan-Mannan Particles

Briefly, the process for producing the glucan particles (GPs) involves the extraction and purification of the alkali-insoluble glucan particles from the yeast or fungal cell walls. The structure-function properties of the glucan particle preparation depend directly on the source from which it is obtained and also from the purity of the final product. The source of glucan particles can be yeast or other fungi, or any other source containing glucan having the properties described herein. In certain embodiments, yeast cells are a preferred source of glucans. The yeast strains employed in the present process can be any strain of yeast, including, for example, S. cerevisiae, S. delbrueckii, S. rosei, S. microellipsodes, S. carlsbergensis, S. bisporus, S. fennentati, S. rouxii, Schizosaccharomyces pombe, Kluyveromyces polysporus, Candida albicans, C. cloacae, C. tropicalis, C. utilis, Hansenula wingei, H. ami, H. henricii, H. americana, H. canadiensis, H. capsulata, H. polymorpha, Pichia kluyveri, P. pastoris, P. polymorpha, P. rhodanensis, P ohmeri, Torulopsis bovin, and T glabrata. Alternatively, mutant yeast strains can be employed.

The yeast cells may be produced by methods known in the art. Typical growth media comprise, for example, glucose, peptone and yeast extract. The yeast cells may be harvested and separated from the growth medium by methods typically applied to separate the biomass from the liquid medium. Such methods typically employ a solid-liquid separation process such as filtration or centrifugation. In the present process, the cells are preferably harvested in the mid-to late logarithmic phase of growth, to minimize the amount of glycogen and chitin in the yeast cells. Glycogen, chitin and protein are undesirable contaminants that affect the biological and hydrodynamic properties of the glucan particles.

Preparation of glucan particles involves treating the yeast with an aqueous alkaline solution at a suitable concentration to solubilize a portion of the yeast and form an alkali-hydroxide insoluble glucan particles having primarily β(1,6) and β(1,3) linkages. The alkali generally employed is an alkali-metal hydroxide, such as sodium or potassium hydroxide or an equivalent. The starting material can comprise yeast separated from the growth medium.

The treating step is performed by extracting the yeast in the aqueous hydroxide solution. The intracellular components and, optionally, the mannan portion, of the cell are solubilized in the aqueous hydroxide solution, leaving insoluble cell wall material which is substantially devoid of protein and having substantially unaltered β(1,6) and β(1,3) linked glucan. The intracellular constituents are hydrolyzed and released into the soluble phase. The conditions of digestion are such that at least in a major portion of the cells, the three dimensional matrix structure of the cell walls is not destroyed. In particular circumstances, substantially all the cell wall glucan remains unaltered and intact.

In certain embodiments, the aqueous hydroxide digestion step is carried out in a hydroxide solution having initial normality of from about 0.1 to about 10.0. A preferred aqueous hydroxide solution is sodium hydroxide. The digestion can be carried out at a temperature of from about 20° C. to about 121° C., for example, at about 70° C. to about 100° C. with lower temperatures requiring longer digestion times. When sodium hydroxide is used as the aqueous hydroxide, the temperature can be about 70° C., 80° C., 90° C. or about 100° C. and the solution has an initial normality of from about 0.75 to about 1.5.

Generally from about 10 to about 500 grams of dry yeast per liter of hydroxide solution is used. In certain embodiments, the aqueous hydroxide digestion step is carried out by a series of contacting steps so that the amount of residual contaminants such as proteins are less than if only one contacting step is utilized. In certain embodiments, it is desirable to remove substantially amounts of protein material from the cell. Additional extraction steps are preferably carried out in a mild acid solution having a pH of from about 2.0 to about 6.0. Typical mild acid solutions include hydrochloric acid, sodium chloride adjusted to the required pH with hydrochloric acid and acetate buffers. Other typical mild acid solutions are in sulfuric acid and acetic acid in a suitable buffer. This extraction step is preferably carried out at a temperature of from about 20° C. to about 100° C. The digested glucan particles can be, if necessary or desired, subjected to further washings and extraction to reduce the protein and contaminant levels. After processing the product pH can be adjusted to a range of about 6.0 to about 7.8.

The glucan particles can be further processed and/or further purified, as desired. For example, the glucan can be dried to a fine powder (e.g., by drying in an oven); or can be treated with organic solvents (e.g., alcohols, ether, acetone, methyl ethyl ketone, chloroform) to remove any traces or organic-soluble material, or retreated with hydroxide solution, to remove additional proteins or other impurities that may be present.

In exemplary methods, about 100 g of yeast, e.g., Bakers yeast, are suspended in about 1 L 1M NaOH and heated to about 80° C. for about 1 hour. Following centrifugation, the insoluble material is suspended in about 1 L of water and the pH adjusted to about 4-5 with HCl and incubated at about 55° C. for about 1 hour. Water and solvent washes can be carried out about 1 to 5 times.

### III. Delivery Peptides

Peptides used in the pcGPs of the invention, e.g., DPs or sDPs, are selected based, at least in part, on their ability to form complexes with nucleic acid payload molecules, e.g., siRNAs, and facilitate transfer of same into cells or intracellular compartments. Preferably, the complexes formed between nucleic acid, e.g., siRNA and peptide, are non-covalent complexes which appear to be best-suited for siRNA delivery with a more significant biological response.

These non-covalent conjugates can be formed through either electrostatic or hydrophobic interactions. Using this method, cargoes or payloads such as nucleic acids, e.g., siRNAs, can be efficiently delivered while maintaining full biological activity. Preferred peptides form highly stable complexes with siRNA and exhibit a low degradation rate and can be easily functionalized for specific targeting.

### A. Peptides

The present invention features a variety of peptides, e.g., DPs or sDPs, having potential effectiveness in nucleic acid delivery, in particular, siRNA delivery, once covalently conjugated to glucan shells. Preferably, the peptides do not elicit an immune response, e.g., elicit antibody production, when used in therapeutic or in vivo delivery systems of the invention, but do effectively bind siRNA and deliver it for effective gene silencing. Preferred peptides for use in the pcGPs of the invention include short, synthetic peptides having the ability to form reversible (non-covalent) complexes with nucleic acid, e.g., siRNA, payload, and/or facilitate endosomal processing of said payload. Preferred peptides are between 5 and 30 amino acids in length, for example, 5-10, 5-15, 5-20, 5-25, 10-15, 10-20, 10-25, 15-20, or 15-25 amino acids in length. Without being bound in theory, it is believed that the length of the peptides, e.g., DPs, can contribute to undesired immunological responses, with longer peptides having greater immune-mediating potential. Accordingly, in preferred aspects, the invention features use of “short delivery peptides (“sDPs”) which are between 5 and 20 amino acids in length, for example, 5-10, 5-15, 5-20, 10-15, 10-20, or 15-20 amino acids in length, for example, 10, 11, 12, 13, 14, 15, 16, 17 18, 19 or 20 amino acids in length. Preferably, the peptides have the ability to facilitate or promote endosomal release of nucleic acid, e.g., siRNA, payloads.

The delivery peptides of the invention typically have an amino acid composition that either contains a high relative abundance of positively charged amino acids making the peptides cationic in nature or have sequences that contain an alternating pattern of polar/charged amino acids and non-polar, hydrophobic amino acids. These two types of structures are referred to as “cationic” (or “polycationic”) and “amphipathic”, respectively. In another aspect, the delivery peptides of the invention have sequences containing two or more histidines, e.g., 2-20, 2-16, 2-10, 2-8 or 2-6 histidines. Preferably such peptides consist of only histidines. This type of structure is referred to as “polyhistidine”.

Representative classes of peptides having potential applications in the technology of the invention along with the amino acid sequences of same are listed below.

1. Amphipathic Peptides

In a preferred aspect of the invention, the delivery peptides used for conjugation to YCWPs (e.g., GPs) of the invention are amphipathic in nature. The term “amphipathic” means that the peptide contains both polar (water-soluble or “water-loving”) and nonpolar (non water-soluble) portions or amino acids in its sequence. Preferred peptides comprise or consist of a combination of non-polar and polar amino acids arranged in pattern, e.g., an alternating pattern, such that the amino acids form a helical structure in which polar residues make up a weakly basic “face” of the peptide and non-polar (or hydrophobic) residues make up a lipophilic “face” of the structure. Amino acids of the weakly basic “face” can include histidines (H) or lysine (K). Amino acids of the lipophilic “face” can include leucine (L). Such peptides can facilitate endocytosis-mediated delivery of cargoes or payloads, in particular, nucleic acid payloads, e.g., siRNAs.

As used herein, the term “endocytosis” refers to the cellular process of invagination and pinching off of the plasma membrane of a cell to form an enclosed vesicle (an endosome) within the cytosolic compartment of the cell, with said endosome being subsequently acidified by proton pumps embedded in the endosomal membrane. This invagination process results in engulfment of substances bound to the outer face of the plasma membrane and/or substances present in the extracellular medium. The term ‘Endocytosis” as used herein can also include related processes, such as pinocytosis and potocytosis, that accomplish a similar engulfment followed by acidification.

The delivery peptides of the invention, e.g., amphiphilic peptides, can form a two-face structure (i.e., 3-dimensional structure), with one face composed predominantly (80% to 100%) of aliphatic lipophilic amino acids (e.g. leucines), and another face composed predominantly (80% to 100%) of basic amino acids (e.g., histidines), preferably, with at least 70% of the amino acids of the weak-base face being histidines. At neutral pH, such as in YCWPs (e.g., GPs) or extracellular medium containing same, or within the cytosol of cells, the delivery peptides can exist in a state which binds but does not permeabilize membranes, while at acidic pH, such as within endosomes, the delivery peptides can reversibly convert to a polycationic state effective to permeabilize cell membranes.

The amphipathic peptides of the invention typically have a length of about 5 to about 30 residues, e.g., about 5 to about 10 residues, about 5 to about 15 residues, about 5 to about 20 residues, about 5 to about 25 residues, about 10 to about 15 residues, about 10 to about 20 residues, about 10 to about 25 residues, about 15 to about 20 residues or about 15 to about 25 residues. In particular exemplary embodiments, the amphipathic peptides of the invention typically have a length of about 5 to about 20 residues, e.g., about 5 to about 10 residues, about 5 to about 15 residues, about 5 to about 20 residues, about 10 to about 15 residues, about 10 to about 20 residues, or about 15 to about 20 residues, for example, 10, 11, 12, 13, 14, 15, 16, 17, 18, 19 or 20 residues. The “about” when referencing peptide length means plus or minus 1 or 2 residues to or from the referenced integer or number of residues. Of exemplary interest are peptides which are modified, e.g., shortened, versions of the amphipathic peptides described in U.S. Pat. No. 7,084,248. For example, shortened versions of peptides having the following sequences:

(SEQ ID NO: 1)

LHHLLHHLLHHLHHLLHHLHHLLHHL,

(SEQ ID NO: 2)

LHKLLHHLLHKLHHLLHKLHHLLHHL,
or

(SEQ ID NO: 3)

LHKLLHHLLHHLHKLLHHLHHLLHKL

can be used in the methods and compositions of the instant invention. Details regarding said peptides can be found, for example, in U.S. Pat. No. 7,084,248.

Also of particular interest are peptides comprising or consisting of variations of lipophillic or hydrophobic residues (e.g., leucine (L)) and basic or weakly basic residues (e.g., histidine (H) or lysine (K)) In exemplary embodiments, the peptides have about 50% lipophillic or hydrophobic residues (e.g., leucine (L)) and 50% basic or weakly basic residues (e.g., histidine (H) or lysine (K)) In some embodiments, the peptides have about 40% lipophillic or hydrophobic residues (e.g., leucine (L)) and 60% basic or weakly basic residues (e.g., histidine (H) or lysine (K)) In other embodiments, the peptides have about 60% lipophillic or hydrophobic residues (e.g., leucine (L)) and 40% basic or weakly basic residues (e.g., histidine (H) or lysine (K)) In preferred amphipathic peptides of the invention, lipophillic or hydrophobic residues are patterned in pairs, with several alternating pairs of lipophillic or hydrophobic residues, optionally with single lipophillic or hydrophobic residues occurring between some pairs of like residues within the peptides. This pattern allows the amphipathic peptides to form a helical structure in which polar residues make up a weakly basic “face” of the peptide and non-polar (or hydrophobic) residues make up a lipophilic “face” of the structure. Amino acids of the weakly basic “face” can include histidine (H) or lysine (K). Preferably, about 65-95% (e.g., about 70-95%, about 75-95%, about 75-95%, about 80-95%, about 85-95% or about 90-95%) of the residues of said amphipathic peptides are within pairs, adjacent pairs or pairs separated by, for example, a single residue of an alternate class type. In the above exemplary embodiments, as lysine is more basic, such residues should preferably constitute no greater than 10-25%, e.g., no greater than 15%, 20% or 25% of the total number of residues of the peptide (the remaining basic residues being, for example, H). An exemplary formula for the above amphipathic peptides is [(A)ₙ₌₁₋₂(B)ₙ₌₁₋₂]ₙ₌₅₋₃₀. Exemplary peptides are as follows:

(SEQ ID NO: 1)

H₂N-LHHLLHHLLHHLHHLLHHLHHLLHHL-COOH,

(SEQ ID NO: 3)

H₂N-LHKLLHHLLHHLHKLLHHLHHLLHKL-COOH

(SEQ ID NO: 2)

H₂N-LHKLLHHLLHKLHHLLHKLHHLLHHL-COOH

(SEQ ID NO: 4)

H₂N-LHHLLHHLLHHLHHL-COOH

(SEQ ID NO: 5)

H₂N-HHLLHHLHHLLHHL-COOH

(SEQ ID NO: 6)

H₂N-LHLLHHLLHHLHHL-COOH,

(SEQ ID NO: 7)

H₂N-LHHLLHLLHHLLHHL-COOH,

(SEQ ID NO: 8)

H₂N-LHKLLHHLLHHLHK-COOH

(SEQ ID NO: 9)

H₂N-LHKLLHHLHHLLHKL-COOH

(SEQ ID NO: 10)

H₂N-KLHHLLHKLHHLLHH-COOH

(SEQ ID NO: 11)

H₂N-HLHLLHHLLHH-COOH,

(SEQ ID NO: 12)

H₂N-LHLLHHLLHH-COOH,

(SEQ ID NO: 13)

H₂N-LHKLLHHLLHKLHHL-COOH

(SEQ ID NO: 14)

H₂N-LHLLHH-COOH,

(SEQ ID NO: 15)

H₂N-LHHLL-COOH,

(SEQ ID NO: 16)

H₂N-LHKLL-COOH

Exemplary amphipathic peptides have standard amino terminal and/or carboxy-terminal ends, but are readily amenable to any of the conjugation chemistries described below (or other art-recognized conjugation chemistries.) Exemplary peptide conjugation is achieved through N-terminal and/or side chain amino groups, e.g., those found in lysine

Preferred amphipathic peptides are functionalized at the C-terminus, for example, to include a C-terminal amide, —COHN2. Without being bound in theory, the amide-functionalized C-terminus converts it from an anionic carboxyl to a non-ionic amide. This can potentially affect the delivery efficiency of the particles (pcGPs).

2. Histidine or Polyhistidines

In another aspect, the delivery peptides of the invention have sequences containing two or more histidines, e.g., 2-20, 2-16, 2-10, 2-8 or 2-6 histidines. Preferably such peptides consist of only histidines. This type of structure is referred to as “polyhistidine”. In preferred embodiments, such polyhistidine peptides have at least 2×His and no more that 15×His, e.g., 2 xHis, 6 xHis, 8 xHis, 10 xHis, 12 xHis, 14 xHis, or 16 xHis. Exemplary peptides are as follows:

(SEQ ID NO: 17)

H₂N-HH-COOH,

(SEQ ID NO: 18)

H₂N-HHHHHH-COOH,

(SEQ ID NO: 19)

H₂N-HHHHHHHH-COOH

(SEQ ID NO: 20)

H₂N-HHHHHHHHHHHHHHH-COOH

Exemplary polyhistidine peptides have standard amino terminal and/or carboxy-terminal ends, but are readily amenable to any of the conjugation chemistries described below (or other art-recognized conjugation chemistries.) Exemplary peptide conjugation is achieved through N-terminal amino groups.

Preferred polyhistidine peptides are functionalized at the C-terminus, for example, to include a C-terminal amide, —COHN2. Without being bound in theory, the amide-functionalized C-terminus converts it from an anionic carboxyl to a non-ionic amide. This can potentially affect the delivery efficiency of the particles (pcGPs).

In exemplary embodiments, 15 xHis peptides are featured. Peptides comprising or consisting of modified histidine are also within the scope of the invention. For example, peptides comprising or consisting of methyl-histidine are exemplary peptides according to the instant invention (e.g., H₂N-(3-Methylhistidine)₁₅-CONH₂).

3. Leucine or Polyleucines

In another aspect, the delivery peptides of the invention have sequences containing two or more leucines, e.g., 2-20, 2-16, 2-10, 2-8 or 2-6 leucines. Preferably such peptides consist of only leucines. This type of structure is referred to as “polyleucine”. In preferred embodiments, such polyleucine peptides have at least 2×Leu and no more that 15×Leu, e.g., 2 xLeu, 6 xLeu, 8 xLeu, 10 xLeu, 12 xLeu, 14 xLeu, or 16 xLeu. Exemplary peptides are as follows:

(SEQ ID NO: 21)

H₂N-LL-COOH,

(SEQ ID NO: 22)

H₂N-LLLLL-COOH,

(SEQ ID NO: 23)

H₂N-LLLLLLLLLL-COOH

(SEQ ID NO: 24)

H₂N-LLLLLLLLLLLLLLL-COOH

Exemplary polyleucine peptides have standard amino terminal and/or carboxy-terminal ends, but are readily amenable to any of the conjugation chemistries described below (or other art-recognized conjugation chemistries.) Exemplary peptide conjugation is achieved through N-terminal amino groups.

Preferred polyleucine peptides are functionalized at the C-terminus, for example, to include a C-terminal amide, —COHN2. Without being bound in theory, the amide-functionalized C-terminus converts it from an anionic carboxyl to a non-ionic amide. This can potentially affect the delivery efficiency of the particles (pcGPs).

In exemplary embodiments, 5 xLeu, 10×Leu or15 xLeu peptides are featured.

4. Cationic Peptides

It has been found that cationic peptides have the propensity to penetrate through a cellular membrane due to a transmembrane potential difference across the membrane. Based at least in part on this activity, certain cationic peptides are useful in the pcGPs of the invention, including, but not limited to, polyarginine peptides, cell-penetrating peptides (or protein transduction domains, PTDs), and other synthetic cationic peptides. Exemplary cationic peptides are as follows:

(SEQ ID NO: 25)

Penetratin - RQIKIWFQNRRMKWKK

(SEQ ID NO: 26)

Transportan - LIKKALAALAKLNIKGLLYGASNLTWG

(SEQ ID NO: 27)

EB1 - LIRLWSHLIHIWFQNRRLKWKKK

(SEQ ID NO: 28)

TAT - GRKKRRQRRRPPQ

(SEQ ID NO: 29)

MPG - GALFLGFLGAAGSTMGAWSQPKKKRKV

(SEQ ID NO: 30)

CADY - GLWRALWRLLRSLWRLLWRA

(SEQ ID NO: 31)

MAP - KLALKLALKALKAALKLA

(SEQ ID NO: 32)

Polyarginine - RRRRRRRRR

(SEQ ID NO: 33)

bPrPp - MVKSKIGSWILVLFVAMWSDVGLCKKRPKP

Truncated, slightly longer and/or slightly modified versions of said sequences are also contemplated for use in the invention, e.g., peptides differing in no greater than 3, 2 or 1 residue in sequence as compared to the above described exemplary peptides and/or differing in a length of about 1, 2, 3, or 4 residues as compared to the above described exemplary peptides. Exemplary cationic peptides have standard amino terminal and/or carboxy-terminal ends, but are readily amenable to any of the conjugation chemistries described below (or other art-recognized conjugation chemistries.)

### B. Conjugation Chemistries

In exemplary aspects the peptide is conjugated to a moiety in the yeast cell wall particle, e.g., GP or GS using a linker moiety. Preferably, the peptide is weakly conjugated to oligosaccharide component(s) in the yeast cell wall particle, e.g., GP or GS. The linker moiety can be covalently bonded to both YCWP, e.g., GP/GS, and delivery peptide. Linker design can impart several properties in the overall function of the conjugate. Typically, linkers are low molecular weight, bifunctional molecules that provide a chemical means to connect functional groups between the YCWP, e.g., GP/GS and the delivery peptide.

A variety of chemistries can be used to attach the peptides to the glucan particles. The peptide can be attached to the particles via either non-degradable or degradable linkages. Degradable linkages can be responsive to such stimuli including but not limited to pH, reductive environment, and enzymes.

Representative degradable linkages include, but are not limited to, hydrazone linkages, acetal linkages, ketal linkages, thioketal linkages, disulfide linkages, ester linkages, orthoester and anhydride linkages.

Representative non-degradable linkages include, but are not limited to, amine linkages, amide linkages, carbonate linkages, carbamate linkages, ether linkages, thioether linkages, oxime and triazole linkages.

Preferred linker functionalities are shown in Table 1.

TABLE 1
Linkers & their functionality

| Moiety | Name | Type |
|---|---|---|
| R₁R₂C═NNH₂ | Hydrazone | Degradable |
| R₁R₂C═NNHR₃ | | |
| R₁C(H)(OR₂)(OR₃) | Acetal | Degradable |
| R₁C(R₂)(OR₃)(OR₄) | Ketal | Degradable |
| R₁C(R₂)(SR₃)(SR₄) | Thioketal | Degradable |
| R₁—S—S—R₂ | Disulfide | Degradable |
| R₁CO(OR₂) | Ester | Degradable |
| R₁C(OR₂)₃ | Orthoester | Degradable |
| R—NH—R | Amine | Stable |
| or R₁N(R₂)(R₃) | | |
| R—CO—NH—R | Amide | Stable/Degradable |
| or R₁—CO—N(R₂)(R₃) | | |
| R₁O—CO(OR₂) | Carbonate | Stable |
| R₁O—CON(R₂)(R₃) | Carbamate | Stable |
| R—O—R | Ether | Stable |
| R—S—R | Thioether | Stable |
| R₁R₂C═N—OR₃ | Oxime | Stable |
| 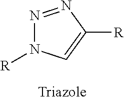 | Triazole | Stable |

Preferred linkers are one of two main types of linkers, stable or degradable. Conjugates with stable linkers are preferable selected to maintain the biochemical function of the YCWP, e.g., GP/GS and peptide within the conjugate. Selection of linker is preferably such that one does not hinder the activity of either the YCWP, e.g., GP/GS or the conjugated peptide.

Degradable linkers can be categorized into two types: pH or chemically degradable and enzymatically degradable. Selection of the linker must consider the above parameters and a synthetic strategy to facilitate efficient assembly of the whole conjugate. Hydrazone moieties have been used as pH-degradable linkers, taking advantage of the pH difference between the plasma and intracellular matrix. The hydrazone group undergoes hydrolysis in the mildly acidic conditions inside the cell, but remains relatively stable in the neutral pH conditions found in the bloodstream. Disulfide moieties employ a chemically cleavable linking strategy, relying on changes in glutathione concentration to release the drug. The concentration of glutathione inside the cell is approximately 1,000-fold higher than in the bloodstream. The higher thiol concentration inside the cell provides for a thiol-disulfide exchange and the corresponding release of the warhead. In certain embodiments, more than one linker can be used to conjugate peptides to YCWP, e.g., GP/GS. Certain linkers may also be stable or degradable, depending on the environment. For example, amide linkers are more stable than certain other degradable linkers listed above, however peptide (of amide) bonds can be enzymatically cleaved inside cells, for example, cells in vivo.

Exemplary amphipathic peptides modified for conjugation to YCWPs (e.g., GPs) of the invention are set forth below:

(SEQ ID NO: 34)

H₂N-LHHLLHHLLHHLHHLLHHLHHLLHHL-CONH₂,

(SEQ ID NO: 35)

H₂N-LHKLLHHLLHHLHKLLHHLHHLLHKL-CONH₂

(SEQ ID NO: 36)

H₂N-LHKLLHHLLHKLHHLLHKLHHLLHHL-CONH₂

(SEQ ID NO: 37)

H₂N-LHHLLHHLLHHLHHL-CONH₂

(SEQ ID NO: 38)

H₂N-HHLLHHLHHLLHHL-CONH₂

(SEQ ID NO: 39)

H₂N-LHLLHHLLHHLHHL-CONH₂,

(SEQ ID NO: 40)

H₂N-LHHLLHLLHHLLHHL-CONH₂,

(SEQ ID NO: 41)

H₂N-LHKLLHHLLHHLHK-CONH₂

(SEQ ID NO: 42)

H₂N-LHKLLHHLHHLLHKL-CONH₂

(SEQ ID NO: 43)

H₂N-KLHHLLHKLHHLLHH-CONH₂

(SEQ ID NO: 44)

H₂N-HLHLLHHLLHH-CONH₂,

(SEQ ID NO: 45)

H₂N-LHLLHHLLHH-CONH₂,

(SEQ ID NO: 46)

H₂N-LHKLLHHLLHKLHHL-CONH₂

(SEQ ID NO: 47)

H₂N-LHLLHH-CONH₂,

(SEQ ID NO: 48)

H₂N-LHHLL-CONH₂,

(SEQ ID NO: 49)

H₂N-LHKLL-CONH₂

The exemplary peptides above, modified for conjugation to YCWP, e.g., GP/GS, are representative of the larger class of peptides which can include any of the peptides described infra in subsection IIIA, combined with any of the conjugation chemistries described in subsection IIIB, infra.

Because peptides are being conjugated to components of YCWP, e.g., GP/GS, for example, to the oligosaccharide component of glucan shells, chemical treatment of the particles may be desirable as part of the conjugation process. For example, conjugation via amide linkage may be preceded by mild oxidation of YCWPs prior to reduction in the presence of peptide. The skilled artisan will readily be able to adapt standard conjugation chemistries for use with the YCWPs of the invention.

A variety of methods for linking one molecule to another via the linkers listed above are well known in the art, such as those described in Bioconjugate Techniques (Hermanson, 1996).

Hydrazone

An acid-degradable hydrazone linkage can be formed by using hydrazides or hydrazines. The reaction can be performed with YCWPs (e.g., GSs/GPs) (using the latent aldehydes at the reducing end of the polysaccharide) or GSs that have been oxidized with sodium periodate prior to the reaction. The GS is resuspended in dd-H₂O by sonication. Borate buffer and the hydrazide of interest (R—CO—NHNH₂) are added to the YCWP (e.g., GS/GP) suspension and the reaction is allowed to proceed overnight at 37° C. The modified YCWP (e.g., GS/GP) is isolated by centrifugation and washed thoroughly with dd-H₂O.

Disulfide

Several methods of linking a molecule to a polysaccharide via a disulfide linker are previously reported. For example, an amine-modified YCWP (e.g., GS/GP) can be reacted with N-Succinimidyl 3-(2-pyridyldithio)-propionate (SPDP, Pierce) to form a pyridyl-disulfide activated particle. This activated YCWP (e.g., GS/GP) can then be reacted with a thiol-containing peptide to create a disulfide linkage. Alternatively, oxidized YCWP (e.g., GS/GP) can be reacted with 3-(2-Pyridyldithio)propionyl hydrazide (PDPH, Pierce) following the procedure described above to form a hydrazone linkage. The pyridyl-disulfide activated particle can then be reacted with a thiol-containing peptide to form a disulfide linkage.

Oxime

A stable oxime linkage can be formed following a procedure similar to that described in Beaudette et al., J. Am. Chem. Soc. (2009) 131, 10360-10361. Reaction of the reducing end aldehyde in YCWPs (e.g., GSs/GPs) (or aldehydes formed through periodate oxidation of the GS) with alkoxyamine reagents yields oxime conjugates. GP/GS (or oxidized GP/GS) are resuspended in a solution containing an aminooxyacetyl-peptide (R—ONH₂). After 2 days at room temperature under gentle agitation, the particles are washed thoroughly.

Triazole

Triazole linkages can be formed on YCWPs (e.g., GSs/GPs) by first forming azide-modified GPs/GSs followed by chemoselective coupling with alkyne-functionalized molecules. Azide-modified GPs/GSs can be prepared following literature procedures (Cui et al., Bioconjugate Chem. (2011) 22, 949-957; Hasegawa et al. Carbohydrate Res. (2006) 341, 35-40). GPs/GSs can be modified using carbonyldiimidazole-activated azido-triethylene glycol (azido-TEG) to afford azido-GP/GS. Alternatively, activation of the primary hydroxyl groups with triphenylphosphine followed by bromination with carbon tetrabromide yields Br-GP/GS. Subsequent azidation using sodium azide affords the azide-modified GPs/GSs. Azido-GPs/GSs can then be functionalized by reaction with alkynes via the ligand-assisted copper-catalyzed azide-alkyne cycloaddition (CuAAC).

Amine

Reductive amination chemistry (as featured in the Examples, infra) can be used to prepare the particles (e.g., pcGPs or amGPs) of the invention. An alternative method uses epibromohydrin to activate the particles followed by amination.

Epibromohydrin Reaction

GP/GS are resuspended in dd-H₂O and 1 M NaOH. Epibromohydrin is added to form a 10% epibromohydrin solution. The reaction is allowed to proceed overnight at 37° C. The activated GP/GS are washed thoroughly and amine is added. The reaction is allowed to proceed overnight at 37° C. under gentle agitation. The amine-modified GP/GS are isolated by centrifugation and washed thoroughly. Peptide-conjugated YCWPs, e.g., pcGPs or amGPs, of the invention can be stored in solution or in dry form, for example, flash-frozen with residual solution, e.g., water, being removed by lyophilization. The latter preparations are particularly suitable for inclusion in kits, for example, kits including peptide-conjugated YCWPs, e.g., pcGPs, or amGPs, and instructions for use with desired siRNAs by the end-user. Alternatively, YCWPs, e.g., GPs/GSs can be included within kits which include peptides, e.g., delivery peptides, optionally further including instructions for use in making pcGPs/GSs or amGPs/GSs. These kits can further include instructions for use with desired siRNAs by the end-user.

### IV. Small Molecule Amines

Preferred aspects of the invention feature “amine-modifed glucan particles” (“amGPs”). In exemplary embodiments, amGPs feature yeast cell wall particles (GPs/GSs) having conjugated, i.e., chemically conjugated thereto, a small molecule amine (an organic compound comprising amine or amino group(s) of a size <900 Daltons) which imparts a desired functionality to said “amine modified glucan particle” (“amGP”). In preferred aspects of the invention, the small molecule amine facilitates delivery of substances capable of complexing with or loading into or onto the amGPs into the cytosol of cells via an endocytosis-mediated process when said particles are contacted with cells, for example, mammalian cells. Exemplary “small molecule amines” are low molecular weight (<900 Daltons) organic compounds comprising, or preferably consisting of carbon, nitrogen, hydrogen and oxygen. In preferred embodiments, such “small molecule amines” are small in nature, e.g., 20-100 Dalton (Da), 40-100 Da, 50-100 Da, 50-200 Da, 100-200 Da, or 100-500 Da. Exemplary “small molecule amines” include aliphatic chains containing primary, secondary and/or tertiary amines, e.g., straight or branched chains, or alicyclic chains, saturated or unsaturated chains, etc., and also include chains comprised primarily of carbon but optionally comprising one or more other elements, for example, oxygen, nitrogen, sulfur, chloride, and the like. Exemplary “small molecule amines” also include aromatic chains containing primary, secondary and/or tertiary amines, e.g., aryl amines, heteroaryl amines (e.g., one or more ring atoms selected from elements such as oxygen, sulfur, nitrogen, etc.) and the like. The term “amine-modified glucan particle” (“amGP”) can be used interchangeably herein with the term “amine-modified glucan particle” (“amGP”).

Several exemplary small molecule amines are set forth in Table 2.

TABLE 2

| | |
|---|---|
| Straight Chains | 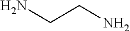 |
| | 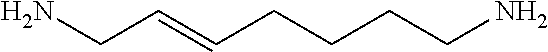 |
| | 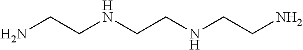 |
| | 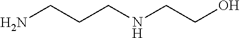 |
| | 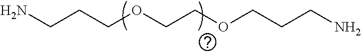 |
| Branched Chains | 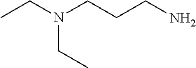 |
| | 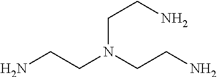 |
| | 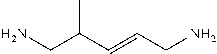 |
| | 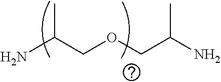 |
| | 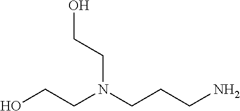 [SMILES: NCCCN(CCO)CCO] |
| | 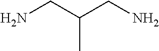 |
| Alicyclic | 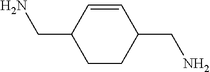 |
| | 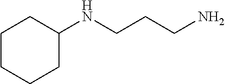 [SMILES: NCCCNC1CCCCC1] |
| | 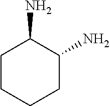 |
| | 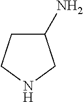 |
| | 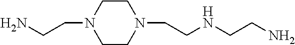 [SMILES: NCCNCCN1CCN(CCN)CC1] |
| | 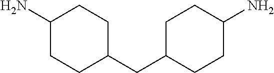 [SMILES: NC1CCC(CC2CCC(N)CC2)CC1] |
| Saturated | 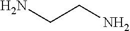 |
| | 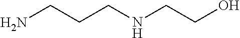 |
| | 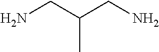 |
| | 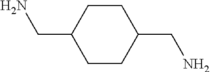 |
| | 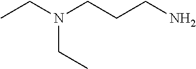 |
| Unsaturated | 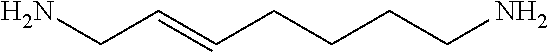 |
| | 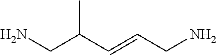 |
| | 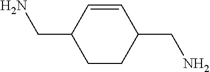 |
| Carbon Chains | 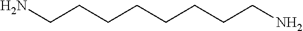 |
| | 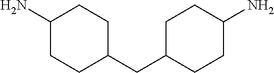 [SMILES: NC1CCC(CC2CCC(N)CC2)CC1] |
| Other Elements | 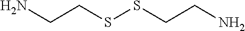 |
| | 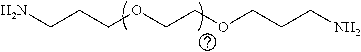 |
| | 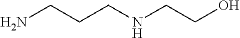 |
| Aryl | 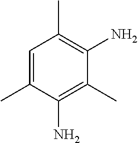 |
| | 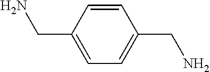 |
| | 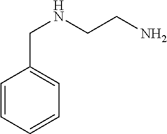 [SMILES: NCCNCc1ccccc1] |
| Heteroaryl | 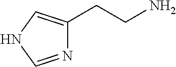 |
| | 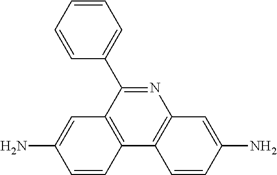 [SMILES: Nc1ccc2c(c1)nc(-c1ccccc1)c1cc(N)ccc12] |

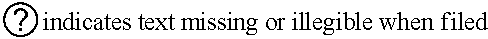

Several exemplary small molecule amines can be depicted structurally, see Table 2, or can be referred to by standard chemical nomenclature. Exemplary nomenclature corresponding to structures set forth in Table 2 is as follows:

Straight chains including: Ethylenediamine; Triethylenetetraamine; 1,10-Diamino-4,7-dioxadecane; N-(2-Hydroxyethyl)-1,3-propanediamine.

Branched chains including: 3-Diethyl aminopropylamine; Tris(2-aminoethyl)amine; 2-Methyl-1,3-propanediamine;

Alicyclic: Cyclohexene-1,4-diyldimethanamine; N-Cyclohexyl-1,3-propanediamine; 1,2-Cyclohexanediamine; 4,4′-Methylenedicyclohexanamine; N1-(2-(4-(2-aminoethyl)piperazin-1-yl)ethyl)ethane-1,2-diamine; 3-Pyrrolidinamine

Saturated: Ethylenediamine; N-(2-Hydroxyethyl)-1,3-propanediamine; 2-Methyl-1,3-propanediamine; 3-Diethylaminopropylamine; Cyclohexane-1,4-diyldimethanamine;

Unsaturated: Cyclohexene-1,4-diyldimethanamine Carbon chain: 1,8-Diaminooctane; 4,4′-Methylenedicyclohexanamine;

Other elements: 1,10-Diamino-4,7-dioxadecane; N-(2-Hydroxyethyl)-1,3-propanediamine; 2-[(2-aminoethyl)disulfanyl]ethan-1-amine; (also includes other elements replace one or more carbons, i.e. oxygen, nitrogen, sulfur, chloride . . . )

Aryl: 2,4,6-Trimethylbenzene-1,3-diamine; N-benzylethane-1,2-diamine; 1,4-Phenylenedimethanamine (all ring atoms are carbon)

Heteroaryl: Histamine; (one or more ring atoms selected from elements other than C, such as O, S, N . . . )

### V. Payload Molecules

The particulate delivery system of the present invention is useful for in vivo or in vitro delivery of payload molecules, in particular, payload molecules having pharmaceutical or therapeutic activity. Exemplary payload molecules include, but are not limited to, nucleic acids, proteins, peptides, enzymes, and smaller molecules, e.g., therapeutic small molecules. Particularly preferred payload molecules are nucleic acid molecules.

Preparations of payload molecules are preferably essentially pure and desirably essentially homogeneous (i.e., free from contaminating molecules observed during synthesis or isolation of payload molecules, etc).

### A. Nucleic Acid Payload Agents

In exemplary embodiments, the compositions of the invention comprise nucleic acid-based payload agents. Exemplary payload agents include, but are not limited to RNA silencing agents (e.g., siRNAs, siRNA-like molecules, miRNAs, shRNAs), other nucleic acids with gene silencing activity (e.g., antisense molecules and/or ribozymes), or nucleic acid constructs (e.g., DNA constructs) encoding said RNA silencing agents and other gene silencing nucleic acids.

1. RNA Silencing Agents

The present invention features RNA silencing agents (e.g. siRNAs, miRNAs, shRNAs) for use in various compositions and methodologies of the invention. The RNA silencing agents comprise an antisense strand (or portions thereof), wherein the antisense strand has sufficient complementarity to a target mRNA to mediate silencing of the mRNA via an RNA-mediated silencing mechanism (e.g., RNAi).

a. siRNA Molecules

An siRNA molecule of the invention is a duplex consisting of a sense strand and complementary antisense strand, the antisense strand having sufficient complementarity to a target mRNA sequence to direct a target-specific RNA silencing mechanism. In preferred embodiments, the antisense strand has sufficient complementarity to the target mRNA to direct RNA interference (RNAi), as defined herein, i.e., the siRNA has a sequence sufficient to trigger the destruction of the target mRNA by the RNA silencing machinery or process. In alternative embodiments, the antisense strand of the siRNA has sufficient complementarity to a target mRNA sequence to direct translation repression of the target mRNA.

In certain embodiments, the siRNA molecule has a length from 5-60 (e.g., about 10-50) or more nucleotides, i.e., each strand comprises 5-60 (e.g., 10-50) nucleotides (or nucleotide analogs). In certain exemplary embodiments, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region, and the other strand is identical or substantially identical to the first strand (e.g., having 5 or fewer (e.g., 1, 2, 3, or 4) mismatches relative to the first strand). In certain particular embodiments, the siRNA molecule has a length of from about 18-25 nucleotides (e.g., 18, 19, 20, 21, 22, 23, 24, or 25 nucleotides). In other particular embodiments, the siRNA molecule has a length of from about 25-30 nucleotides (e.g., 25, 26, 27, 28, 29, or 30 nucleotides). In other particular embodiments, the siRNA molecule has a length of from about 25-35 nucleotides (e.g., 25, 26, 27, 28, 29, 30, 31, 32, 33, 34 or 35 nucleotides). In other embodiments, siRNAs may have shorter or longer lengths. In one embodiment, the siRNA has a length of about 5-15 nucleotides or nucleotide analogs (e.g., 5, 6, 7, 8, 9, 10, 11, 12, 13, 14, or 15 nucleotides) in each strand, wherein one of the strands is sufficiently complementary to a target region. In another embodiment, the siRNA has a length of about 30-35 nucleotides or nucleotide analogs (e.g., 30, 31, 32, 33, 34 or 35 nucleotides in each strand, wherein one of the strands is sufficiently complementary to a target region). In another embodiment, the siRNA has a length of about 30-60 nucleotides or nucleotide analogs (e.g., 35, 40, 45, 50, 55, or 60 nucleotides in each strand, wherein one of the strands is sufficiently complementary to a target region).

In certain embodiments, the strands of the siRNA molecule are of different lengths (e.g., they differ in length by 5 or fewer nucleotides (e.g., 1, 2, 3, or 4)). In other embodiments, the strands of the siRNA molecule are of the same length.

In certain embodiments, the strands of the siRNA molecule aligned such that one or both ends of the siRNA molecule are blunt-ended (i.e., lack an overhang). In other embodiments, the strands of the siRNA molecule are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. In certain embodiments, at least one (preferably both) ends of the duplex comprise a 2 nucleotide overhang (e.g., dTdT overhangs).

Generally, siRNAs can be designed by using any method known in the art, for instance, by using the following protocol:

1. A target mRNA is selected and one or more target sites are identified within said target mRNA. Cleavage of mRNA at these sites results in mRNA degradation, preventing production of the corresponding protein. Polymorphisms from other regions of the mutant gene are also suitable for targeting.

In preferred embodiments, the target sequence comprises AA dinucleotide sequences; each AA and the 3′ adjacent 16 or more nucleotides are potential siRNA targets. In another preferred embodiment, the nucleic acid molecules are selected from a region of the target mRNA sequence beginning at least 50 to 100 nt downstream of the start codon, e.g., of the sequence of the target mRNA. Further, siRNAs with lower G/C content (35-55%) may be more active than those with G/C content higher than 55%. Thus in one embodiment, the invention includes target sequences having 35-55% G/C content, although the invention is not limited in this respect.

2. The sense strand of the siRNA is designed based on the sequence of the selected target site. For example, the sense strand may include about 18 to 25 nucleotides, e.g., 18, 19, 20, 21, 22, 23, 24 or 25 nucleotides. The skilled artisan will appreciate, however, that siRNAs having a length of less than 19 nucleotides or greater than 25 nucleotides can also function to mediate RNAi. For example, in certain embodiments, the sense strand may include about 25 to about 30 nucleotides, e.g., 25, 26, 27, 28, 29, or 30 nucleotides. In other embodiments, the sense strand may include about 30 to about 35 nucleotides, e.g., 30, 31, 32, 33, 34 or 35 nucleotides. Accordingly, siRNAs of such length are also within the scope of the instant invention provided that they retain the ability to mediate RNAi. RNA silencing agents of longer lengths have been demonstrated to elicit an interferon or PKR response in certain mammalian cells which may be undesirable. Preferably the RNA silencing agents of the invention do not elicit a PKR response (i.e., are of a sufficiently short length). However, longer RNA silencing agents may be useful, for example, in cell types incapable of generating a PKR response or in situations where the PKR response has been downregulated or dampened by alternative means.

The siRNA molecules of the invention have sufficient complementarity with the target site such that the siRNA can mediate RNAi. In general, siRNA containing nucleotide sequences sufficiently identical to a portion of the target gene to effect RISC-mediated cleavage of the target gene are preferred. Accordingly, in a preferred embodiment, the sense strand of the siRNA is designed to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment is generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense strand sequence is designed such that nucleotides corresponding to the desired target cleavage site are essentially in the middle of the strand. For example, if a 21-nucleotide siRNA is chosen, nucleotides corresponding to the target cleavage site are at, for example, nucleotide 6, 7, 8, 9, 10, 11, 12, 13, 14, 15 or 16 (i.e., 6, 7, 8, 9, 10, 11, 12, 13, 14, 15 or 16 nucleotides from the 5′ end of the sense strand). For a 22-nucleotide siRNA, nucleotides corresponding to the target cleavage site are at, for example, nucleotide 7, 8, 9, 10, 11, 12, 13, 14, 15 or 16. For a 23-nucleotide siRNA, nucleotides corresponding to the target cleavage site are at, for example, 7, 8, 9, 10, 11, 12, 13, 14, 15 or 16. For a 24-nucleotide siRNA, nucleotides corresponding to the target cleavage site are at, for example, 9, 10, 11, 12, 13, 14, 15 or 16. For a 25-nucleotide siRNA, nucleotides corresponding to the target cleavage site are at, for example, 9, 10, 11, 12, 13, 14, 15, 16 or 17. Moving nucleotides corresponding to an off-center position may, in some instances, reduce efficiency of cleavage by the siRNA. Such compositions, i.e., less efficient compositions, may be desirable for use if off-silencing of a second (non-target) mRNA is detected.

The sense strand is designed such that complementarity exists between the antisense strand of the siRNA and the sense strand. In certain exemplary embodiments, the siRNA is designed such that the strands have blunt ends. In other exemplary embodiments, the siRNA is designed such that the strands have overhanging ends, e.g., overhangs of 1, 2, 3, 4, 5 or more nucleotide at one, or both, ends of the siRNA. Exemplary overhangs are deoxynucleotide overhangs, for example, a dTdT tail.

4. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus in another embodiment, the nucleic acid molecules may have a 3′ overhang of 2 nucleotides, such as TT. The overhanging nucleotides may be either RNA or DNA.

5. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

6. Select one or more sequences that meet your criteria for evaluation.

Further general information about the design and use of siRNA may be found in “The siRNA User Guide,” available at The Max-Plank-Institut für Biophysikalishe Chemie website.

Alternatively, the siRNA may be defined functionally as comprising an antisense or guide strand having a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome. In addition, negative control siRNAs can be designed by introducing a significant number of base mismatches into the sequence.

7. To validate the effectiveness by which siRNAs cleave target mRNAs (e.g., mutant mRNAs), the siRNA may be incubated with target cDNA in a Drosophila-based in vitro mRNA expression system. Radiolabeled with ³²P, newly synthesized mutant target mRNAs are detected autoradiographically on an agarose gel. The presence of cleaved mutant mRNA indicates mRNA nuclease activity. Suitable controls include omission of siRNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

b. siRNA-Like Molecules

In other embodiments, the compositions of the instant invention comprise siRNA-like molecules. siRNA-like molecules of the invention have a sequence (i.e., have a strand having a sequence) that is “sufficiently complementary” to a target mRNA to direct gene silencing either by RNA silencing or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNA silencing is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity in a manner similar to that described below for miRNAs. In one embodiment, where gene silencing by translational repression is desired, at least one non-identical nucleotide is present in the central portion of the complementarity site so that duplex formed by the guide strand of the siRNA-like duplex and the target mRNA contains a central “bulge” (Doench J G et al., Genes & Dev., 2003). In another embodiment, 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further preferred embodiment, a “bulge” is centered at nucleotide positions 12 and 13, referencing the 5′end of the guide strand of the siRNA-like molecule.

c. miRNAs

In certain embodiments, the compositions of the invention comprise miRNAs. miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof.

The miRNA sequence can be similar or identical to that of any naturally occurring miRNA (see e.g. The miRNA Registry; Griffiths-Jones S, Nuc. Acids Res., 2004). Thousands of natural miRNAs have been identified to date and together they are thought to comprise at least 1% of all predicted genes in the genome. An online registry provides a searchable database of all published miRNA sequences (The miRNA Registry at the Sanger Institute website; Griffiths-Jones S, Nuc. Acids Res., 2004). Exemplary, natural miRNAs include lin-4, let-7, miR-10, mirR-15, miR-16, miR-168, miR-175, miR-196 and their homologs, as well as other natural miRNAs from humans and certain model organisms including Drosophila melanogaster, Caenorhabditis elegans, zebrafish, Arabidopsis thalania, mouse, and rat as described in International PCT Publication No. WO 03/029459.

Certain miRNAs, e.g. plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between a miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism. In particular embodiments, the miRNA sequence is that of a naturally-occurring miRNA sequence, the aberrant expression or activity of which is correlated with a miRNA disorder.

Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, may comprise sequence derived from these naturally occurring pre-miRNAs, but are engineered to deliver desired RNA silencing agents (e.g., siRNAs of the invention). For example, by substituting the stem sequences of the pre-miRNA with sequence complementary to the target mRNA, a shRNA is formed. The shRNA is processed by the entire gene silencing pathway of the cell, thereby efficiently mediating RNAi.

In certain embodiments, where post-transcriptional gene silencing by translational repression of the target gene is desired, the miRNA sequence has partial complementarity with the target gene sequence. In exemplary embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

d. Short Hairpin RNA (shRNA) Molecules

In certain embodiments, the compositions of the invention comprise shRNAs. In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway.

The requisite elements of a shRNA molecule include a first portion and a second portion, having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. The shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In exemplary shRNAs, one portion of the duplex stem is a nucleic acid sequence that is complementary (or anti-sense) to the target mRNA. Preferably, one strand of the stem portion of the shRNA is sufficiently complementary (e.g., antisense) to a target RNA (e.g., mRNA) sequence to mediate degradation or cleavage of said target RNA via RNA interference (RNAi). Thus, shRNAs may include a duplex stem with two portions and a loop connecting the two stem portions. The antisense portion can be on the 5′ or 3′ end of the stem. The stem portions of a shRNA are preferably about 15 to about 50 nucleotides in length. Preferably the two stem portions are about 18 or 19 to about 21, 22, 23, 24, 25, 30, 35, 37, 38, 39, or 40 or more nucleotides in length. In preferred embodiments, the length of the stem portions should be 21 nucleotides or greater. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to, and including the entire mRNA). In fact, a stem portion can include much larger sections complementary to the target mRNA (up to, and including the entire mRNA).

The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides. The overhanging nucleotides can include, for example, uracils (Us), e.g., all Us. The loop in the shRNAs can be 2, 3, 4, 5, 6, 7, 8, 9, or more, e.g., 15 or 20, or more nucleotides in length.

A preferred loop consists of or comprises a “tetraloop” sequence. Exemplary tetraloop sequences include, but are not limited to, the sequences GNRA, where N is any nucleotide and R is a purine nucleotide, GGGG, and UUUU.

In certain embodiments, shRNAs of the invention include the sequences of a desired siRNA molecule described supra. In other embodiments, the sequence of the antisense portion of a shRNA can be designed essentially as described above or generally by selecting an 18, 19, 20, 21 nucleotide, or longer, sequence from within the target RNA, for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. This 21 or so nucleotide sequence is used to create one portion of a duplex stem in the shRNA. This sequence can replace a stem portion of a wild-type pre-miRNA sequence, e.g., enzymatically, or is included in a complete sequence that is synthesized. For example, one can synthesize DNA oligonucleotides that encode the entire stem-loop engineered RNA precursor, or that encode just the portion to be inserted into the duplex stem of the precursor, and using restriction enzymes to build the engineered RNA precursor construct, e.g., from a wild-type pre-miRNA.

Engineered RNA precursors include in the duplex stem the 21-22 or so nucleotide sequences of the siRNA, siRNA-like duplex, or miRNA desired to be produced in vivo. Thus, the stem portion of the engineered RNA precursor includes at least 18 or 19 nucleotide pairs corresponding to the sequence of an exonic portion of the gene whose expression is to be reduced or inhibited. The two 3′ nucleotides flanking this region of the stem are chosen so as to maximize the production of the siRNA from the engineered RNA precursor and to maximize the efficacy of the resulting siRNA in targeting the corresponding mRNA for translational repression or destruction by RNAi in vivo and in vitro. In certain embodiments, shRNAs may include miRNA sequences, optionally end-modified miRNA sequences, to enhance entry into RISC.

B. Chemically-Modified RNA Silencing Agents

In certain aspects, the compositions of the invention comprise RNA silencing agents wherein the sense strand and/or antisense strand is modified by the substitution of nucleotides with chemically modified nucleotides. In one embodiment, the sense strand and/or the anti sense strand are modified with one or more internal chemical modifications. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or the antisense strand are modified at the 5′end and/or the 3′ end. In one embodiment, the sense strand and/or the antisense strand are modified at both the 5′end and the 3′ end. As used herein, the term “modified at the end” when used in reference to the 5′ or 3′ ends, refers to any nucleotide within 10 nucleotides of the first and last nucleotide, for example any nucleotide within 7 nucleotides of the first and last nucleotide. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the nucleotides. In yet another embodiment, the sense strand and/or antisense strand is modified by the substitution of all of the nucleotides. Within the RNA silencing agents employed in the invention, as few as one and as many as all nucleotides of the oligonucleotide can be modified. In some embodiments, the RNA silencing agent will contain as few modified nucleotides as are necessary to achieve a desired level of in vivo stability and/or bioaccessibility while maintaining cost effectiveness.

Chemical modifications may lead to increased stability, e.g., increased or enhanced in vivo stability, compared to an unmodified RNA silencing agent or a label that can be used, e.g., to trace the RNA silencing agent, to purify an RNA silencing agent, or to purify the RNA silencing agent and cellular components with which it is associated. Such chemical modifications can also be used to stabilize the first (priming) strand of the siRNA for enhancing RISC activity/RNA silencing responsiveness in a cell (or cell extract or organism) and improve its intracellular half-life for subsequent receipt of the second strand wherein RNA silencing/gene silencing can now progress. Modifications can also enhance properties such as cellular uptake of the RNA silencing agents and/or stability of the RNA silencing agents, can stabilize interactions between base pairs, and can maintain the structural integrity of the antisense RNA silencing agent-target RNA duplex. RNA silencing agent modifications can also be designed such that properties important for in vivo applications, in particular, human therapeutic applications, are improved without compromising the RNA silencing activity of the RNA silencing agents e.g., modifications to increase resistance of, e.g., siRNA or miRNA molecules to nucleases. In certain embodiments, modified siRNA molecules of the invention can enhance the efficiency of target RNA inhibition as compared to a corresponding unmodified siRNA. In some embodiments, modified nucleotides do not affect the ability of the anti sense strand to adopt A-form helix conformation when base-pairing with the target RNA sequence, e.g., an A-form helix conformation comprising a normal major groove when base-pairing with the target RNA sequence.

Chemical modifications generally include end-, sugar-, base- and/or backbone-modifications to the ribonucleotides (i.e., include modifications to the phosphate-sugar backbone).

In one embodiment, the RNA silencing agent of the invention comprises one or more (e.g., about 1, 2, 3, or 4) end modifications. For example, modification at the 5′ end of an siRNA molecule comprises, for example, a 5′-propylamine group. Modifications of the 5′ end may also include 5′ terminal phosphate groups, such as those described by Formula I:

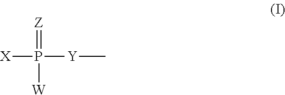

wherein each X and Y is independently O, S, N, alkyl, substituted alkyl, or alkylhalo; wherein each Z and W is independently O, S, N, alkyl, substituted alkyl, O-alkyl, S-alkyl, alkaryl, aralkyl, alkylhalo, or acetyl. In some embodiments, W, X, Y and Z are not all O. Modifications to the 3′ OH terminus of an siRNA molecule can include, but are not limited to, 3′-puromycin, 3′-biotin (e.g., a photocleavable biotin), a peptide (e.g., a Tat peptide), a nanoparticle, a peptidomimetic, organic compounds (e.g., a dye such as a fluorescent dye), or a dendrimer. End modifications may be on the sense strand, on the antisense strand or both. In some embodiments, the 5′ modifications are on the sense strand only.

In another embodiment, the RNA silencing agent of the invention may comprise one or more (e.g., about 1, 2, 3, 4, 5, 6, 7, 8, 9, 10, or more) sugar-modified nucleotides. Exemplary sugar modifications may include modifications represented by Formula II:

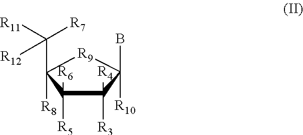

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, or O-aminoacyl, heterocycloalkyl, heterocycloalkaryl, aminoalkylamino, polyalkylamino, substituted silyl; R9 is O, S, CH2, S═O, CHF, or CF2, and B is a nucleosidic base. Sugar-modified nucleotides include, but are not limited to: 2′-fluoro modified ribonucleotides, 2′-OMe modified ribonucleotides, 2′-deoxy ribonucleotides, 2′-amino modified ribonucleotides and 2′-thio modified ribonucleotides. The sugar-modified nucleotide can be, for example, 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine or 2′-amino-butyryl-pyrene-uridine. In one embodiment, the sugar-modified nucleotide is a 2′-fluoro ribonucleotide. In some embodiments, when a 2′-deoxy ribonucleotide is present, it is upstream of the cleavage site referencing the antisense strand or downstream of the cleavage site referencing the antisense strand. The 2′-fluoro ribonucleotides can be in the sense and antisense strands. In some embodiments, the 2′-fluoro ribonucleotides are every uridine and cytidine. In other embodiments, the 2′-fluoro ribonucleotides are only present at the 3′ and 5′ ends of the sense strand, the antisense strand or both.

In another embodiment, the RNA silencing agent of the invention comprises one or more (e.g., about 1, 2, 3, 4, 5, 6, 7, 8, 9, 10, or more) nucleobase-modified nucleotides. Nucleobase-modified nucleotides useful in the invention include, but are not limited to: uridine and/or cytidine modified at the 5-position (e.g., 5-bromo-uridine, 5-(2-amino)propyl uridine, 5-amino-allyl-uridine, 5-iodo-uridine, 5-methyl-cytidine, 5-fluoro-cytidine, and 5-fluoro-uridine), ribo-thymidine, 2-aminopurine, 2, 6-diaminopurine, 4-thio-uridine, adenosine and/or guanosines modified at the 8 position (e.g., 8-bromo guanosine), deaza nucleotides (e.g., 7-deaza-adenosine), O- and N-alkylated nucleotides (e.g., N6-methyl adenosine) and non-nucleotide-type bases (e.g., deoxy-abasic, inosine, N3-methyl-uridine, N6, N6-dimethyl-adenosine, pseudouridine, purine ribonucleoside and ribavirin).

In another embodiment, the RNA silencing agent of the invention comprises one or more (e.g., about 1, 2, 3, 4, 5, 6, 7, 8, 9, 10, or more) backbone-modified nucleotides. For example, backbone modifications may include modifications represented by Formula III:

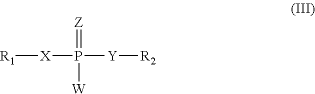

wherein each R1 and R2 is independently any nucleotide as described herein, each X and Y is independently O, S, N, alkyl, or substituted alkyl, each Z and W is independently O, S, N, alkyl, substituted alkyl, O-alkyl, S-alkyl, alkaryl, aralkyl, or acetyl. In some embodiments, W, X, Y, and Z are not all O. Exemplary backbone-modified nucleotides contain a phosphorothioate group or a phosphorodithioate. In another embodiment, a backbone modification of the invention comprises a phosphonoacetate and/or thiophosphonoacetate internucleotide linkage (see for example Sheehan et al., 2003, Nucleic Acids Research, 31, 4109-4118). The backbone-modifications can be within the sense strand, antisense strand, or both the sense and antisense strands. In some embodiments, only a portion of the internucleotide linkages are modified in one or both strands. In other embodiments, all of the internucleotide linkages are modified in one or both strands. In one embodiment, the modified internucleotide linkages are at the 3′ and 5′ ends of one or both strands.

In another embodiment, the siRNA molecule of the invention may comprise one or more (e.g., about 1, 2, 3, 4, 5, 6, 7, 8, 9, 10, or more) crosslinks, e.g., a crosslink wherein the sense strand is crosslinked to the antisense strand of the siRNA duplex. Crosslinkers useful in the invention are those commonly known in the art, e.g., psoralen, mitomycin C, cisplatin, chloroethylnitrosoureas and the like. In one embodiment, the crosslink of the invention is a psoralen crosslink. Preferably, the crosslink is present downstream of the cleavage site referencing the antisense strand, and more preferably, the crosslink is present at the 5′ end of the sense strand.

In another embodiment, the RNA silencing agent of the invention comprises a sequence wherein the antisense strand and target mRNA sequences comprise one or more (e.g., about 1, 2, 3, 4, 5, 6, 7, 8, 9, 10, or more) mismatches. In some embodiments, the mismatch is downstream of the cleavage site referencing the antisense strand, e.g., within 1-6 nucleotides from the 3′ end of the antisense strand. In another embodiment, the nucleic acid molecule, e.g., RNA silencing agent, of the invention is an siRNA molecule that comprises a bulge, e.g., one or more (e.g., about 1, 2, 3, 4, 5, 6, 7, 8, 9, 10, or more) unpaired bases in the duplex siRNA. In some embodiments, the bulge is in the sense strand.

It is to be understood that any of the above combinations can be used in any combination to provide the modified RNA silencing agent of the present invention. For example, in some embodiments, the invention includes an siRNA, wherein the sense strand includes one or more (e.g., about 1, 2, 3, 4, 5, 6, 7, 8, 9, 10, or more) phosphorothioate internucleotide linkages, and/or one or more (e.g., about 1, 2, 3, 4, 5, 6, 7, 8, 9, 10 or more) 2′-deoxy, 2′-O-methyl, and/or 2′-fluoro sugar modifications, and/or one or more (e.g., about 1, 2, 3, 4, 5, 6, 7, 8, 9, 10 or more) base modified nucleotides, and/or an end-modification at the 3′-end, the 5′-end, or both the 3′- and 5′-ends of the sense strand. In some embodiments, the invention includes an siRNA, wherein the antisense strand includes one or more (e.g., about 1, 2, 3, 4, 5, 6, 7, 8, 9, 10, or more) phosphorothioate internucleotide linkages, and/or one or more (e.g., about 1, 2, 3, 4, 5, 6, 7, 8, 9, 10 or more) 2′-deoxy, 2′-O-methyl, and/or 2′-fluoro sugar modifications, and/or one or more (e.g., about 1, 2, 3, 4, 5, 6, 7, 8, 9, 10 or more) base modified nucleotides, and/or an end-modification at the 3′-end, the 5′-end, or both the 3′- and 5′-ends of the antisense strand. In yet other embodiments, the invention includes an siRNA, wherein both the sense strand and the antisense strand include one or more (e.g., about 1, 2, 3, 4, 5, 6, 7, 8, 9, 10, or more) phosphorothioate internucleotide linkages, and/or one or more (e.g., about 1, 2, 3, 4, 5, 6, 7, 8, 9, 10 or more) 2′-deoxy, 2′-O-methyl, and/or 2′-fluoro sugar modifications, and/or one or more (e.g., about 1, 2, 3, 4, 5, 6, 7, 8, 9, 10 or more) base modified nucleotides, and/or an end-modification at the 3′-end, the 5′-end, or both the 3′- and 5′-ends of either or both the sense strand and/or the antisense strand.

Modified RNA silencing agents of the invention (i.e., duplex siRNA molecules) can be modified at the 5′ end, 3′ end, 5′ and 3′ end, and/or at internal residues, or any combination thereof. RNA silencing agent modifications can be, for example, end modifications, sugar modifications, nucleobase modifications, backbone modifications, and can contain mismatches, bulges, or crosslinks. Also included are 3′ end, 5′ end, or 3′ and 5′ and/or internal modifications, wherein the modifications are, for example, cross linkers, heterofunctional cross linkers and the like. RNA silencing agents of the invention also may be modified with chemical moieties (e.g., cholesterol) that improve the in vivo pharmacological properties of the RNA silencing agents.

In certain aspects of the present invention, the chemically modified siRNAs of the present invention are “terminally-modified siRNAs”. That is, the siRNAs are modified at one or both of the 3′ end and the 5′ end of the sense and/or antisense strand. In certain embodiments, the chemically modified siRNAs are modified at both the 3′ end and the 5′ end of both the sense and/or antisense strand. In some embodiments, the 3′ end and/or the 5′ end of the sense and/or antisense strands are end-modified such that 2 or 3 or 4 modified nucleotides are incorporated per end (e.g., within the 5-7 terminal nucleotides, e.g., within the duplex). In some embodiments, the 3′ end and/or the 5′ end of the sense and/or antisense strands are end-modified such that 2 or 3 or 4 2′-fluoro nucleotides, e.g., 2′ fluorocytidine and/or 2′-fluorouracil, are incorporated per end (e.g., within the 5-7 terminal nucleotides, e.g., within the duplex). In some embodiments, the 3′ end and/or the 5′ end of the sense and/or antisense strands are end-modified such that 2 or 3 or 4 internucleotide linkages are phosphorothioate linkages per end (e.g., between the 5-7 terminal nucleotides, e.g., within the duplex). In some embodiments, the modifications include any of the modifications described herein. In other embodiments, the modifications include phosphorothioate linkages. In still other embodiments, the modifications include 2′-sugar modifications. In still other embodiments, the modifications include 2′-fluoro nucleotide modifications. In yet other embodiments, the modifications include both phosphorothioate linkages and 2′-fluoro nucleotide modifications.

In other aspects, RNA silencing agents may be modified according to methods described in the art (Amarzguioui et. al., Nuc. Acids. Res., (2003) 31: 589-95; Chiu and Rana, RNA, (2003), 9: 1034-48; Chiu and Rana, Mol. Cell., (2002), 10: 549-61); Morrissey et al., Nat. Biotech., (2005), 23: 2002-7), each of which is incorporated by reference herein. In one embodiment, the RNA silencing agent may be conjugated to a lipophilic moiety. In one embodiment, the lipophilic moiety is a ligand that includes a cationic group. In another embodiment, the lipophilic moiety is attached to one or both strands of an siRNA. In a preferred embodiment, the lipophilic moiety is attached to one end of the sense strand of the siRNA. In another preferred embodiment, the lipophilic moiety is attached to the 3′end of the sense strand. In certain embodiments, the lipophilic moiety is selected from the group consisting of cholesterol, vitamin E, vitamin K, vitamin A, folic acid, or a cationic dye (e.g., Cy3). In a preferred embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyelithocholic acid, O3-(oleoyl)cholenic acid, dimethoxytrityl, or phenoxazine.

In some embodiments, the RNA silencing agent of the instant invention may also contain a nuclear localization/nuclear targeting signal(s). Such modifications may be made exclusive of, or in addition to, any combination of other modifications as described herein. Nuclear targeting signals include any art-recognized signal capable of effecting a nuclear localization to a molecule, including, for example, NLS signal sequence peptides.

Oligonucleotide RNA silencing agents may be produced enzymatically or by partial/total organic synthesis. In one embodiment, an RNA silencing agent, e.g., siRNA, is prepared chemically. Methods of synthesizing RNA and DNA molecules are known in the art, in particular, the chemical synthesis methods as described in Verma and Eckstein (1998) Annul Rev. Biochem. 67:99-134. RNA can be purified from a mixture by extraction with a solvent or resin, precipitation, electrophoresis, chromatography, or a combination thereof. Alternatively, the RNA may be used with no or a minimum of purification to avoid losses due to sample processing. Alternatively, the RNA molecules, e.g., RNA silencing oligonucleotides, can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strands.

In one embodiment, siRNAs are synthesized either in vivo, in situ, or in vitro. Endogenous RNA polymerase of the cell may mediate transcription in vivo or in situ, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses siRNA from a recombinant construct may be produced by introducing the construct into a zygote, an embryonic stem cell, or another multipotent cell derived from the appropriate organism.

Expression levels of target and any other surveyed RNAs and proteins may be assessed by any of a wide variety of well known methods for detecting expression of non-transcribed nucleic acid, and transcribed nucleic acid or protein. Non-limiting examples of such methods include RT-PCR of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e. a transcribed polynucleotide) from a cell, tissue or organism, and by hybridizing the mRNA/cDNA with a reference polynucleotide which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

C. Other Nucleic Acid Molecules

In other embodiments, a nucleic acid molecule employed in a composition of the invention is a nucleic acid molecule other than an RNA silencing agent. In certain embodiments, said nucleic acid molecules may comprise any of the chemical modifications discussed supra.

1. Antisense Oligonucleotides

In one embodiment, a nucleic acid molecule employed in the invention is an antisense nucleic acid molecule that is complementary to a target mRNA or to a portion of said mRNA, or a recombinant expression vector encoding said antisense nucleic acid molecule. Antisense nucleic acid molecules are generally single-stranded DNA, RNA, or DNA/RNA molecules which may comprise one or more nucleotide analogs. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the target mRNA sequence and accordingly is capable of hydrogen bonding to the mRNA. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Preferably, an antisense nucleic acid is designed so as to be complementary to a region preceding or spanning the initiation codon in the 3′ untranslated region of an mRNA.

Given the known nucleotide sequence of a target mRNA, antisense nucleic acids of the invention can be designed according to the rules of Watson and Crick base pairing. The antisense nucleic acid molecule can be complementary to the entire coding region of an mRNA, but more preferably is antisense to only a portion of the coding or noncoding region of an mRNA. For example, the antisense oligonucleotide can be complementary to the region surrounding the translation start site of a target mRNA. An antisense oligonucleotide can be, for example, about 5, 10, 15, 20, 25, 30, 35, 40, 45, 50, 60, 70, 80, 100, 500, 1000 nucleotides or more in length. In some embodiments, the anti sense oligonucleotide may be as long as, or longer than, the length of the mRNA that is targeted.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, as described supra.

To inhibit expression in cells, one or more antisense oligonucleotides can be used. Alternatively, an antisense nucleic acid can be produced biologically using an expression vector into which all or a portion of a cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the cDNA (or portion thereof) is cloned into the vector in the antisense orientation. The antisense expression vector can be in the form of, for example, a recombinant plasmid, phagemid or attenuated virus.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix.

In one particular embodiment, antisense oligonucleotides may be employed which are complementary to one or more of the RNA silencing agents (e.g., miRNA molecules) described supra. Said anti-miRNA oligonucleotides may be DNA or RNA oligonucleotides, or they may be comprised of both ribonucleotide and deoxyribonucleotides or analogs thereof. In preferred embodiments, said anti-miRNA oligonucleotides comprise one or more (e.g., substantially all) 2′O-methyl ribonucleotides. Such molecules are potent and irreversible inhibitors of miRNA-mediated silencing and are therefore useful for modulating RNA silencing both in vitro and in vivo. In vivo methodologies are useful for both general RNA silencing modulatory purposes as well as in therapeutic applications in which RNA silencing modulation (e.g., inhibition) is desirable. For example, insulin secretion has been shown to be regulated by at least one miRNA (Poy et al. 2004), and a role for miRNAs has also been implicated in spinal muscular atrophy (SMA; Mourelatos et al. 2002).

2. α-Anomeric Nucleic Acid Molecules

In yet another embodiment, a nucleic acid molecule employed in the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). Such a nucleic acid molecule can also comprise a 2′-O-methylribonucleotide (Inoue et al. (1987) Nucleic Acids Res. 15:6131-6148) or a chimeric RNA-DNA analogue (Inoue et al. (1987) FEBS Lett. 215:327-330).

3. Ribozymes

In still another embodiment, a nucleic acid molecule employed in the invention is a ribozyme. Ribozymes are catalytic RNA molecules having extensive secondary structure and which are intrinsically capable of cleaving a single-stranded nucleic acid, such as an mRNA, to which they have a complementary region. Thus, ribozymes can be used to catalytically cleave mRNA transcripts to thereby inhibit translation of mRNAs (see e.g., Haselhoff and Gerlach (1988) Nature 334:585-591). A ribozyme having specificity for a target gene can be designed based upon the nucleotide sequence of the cDNA or mRNA of the gene. For example, ribozyme RNA can be constructed in which the nucleotide sequence of the active site is complementary to the nucleotide sequence to be cleaved in a target mRNA. See, e.g., Cech et al. U.S. Pat. No. 4,987,071 and Cech et al. U.S. Pat. No. 5,116,742. Alternatively, a target mRNA can be used to select a catalytic RNA having a specific ribonuclease activity from a pool of RNA molecules. See, e.g., Bartel, D. and Szostak, J. W. (1993) Science 261:1411-1418.

4. Triple Helix Molecules

Alternatively, gene expression can be inhibited by targeting nucleotide sequences complementary to the regulatory region of a target gene to form triple helical structures that prevent transcription of a gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

5. Nucleic Acid Vectors

In other embodiments, a nucleic acid molecule of the invention is a vector, e.g., an expression vector containing a nucleic acid encoding a gene product (or portion thereof, e.g. a protein) or an RNA silencing agent or any other nucleic acid discussed supra. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, adeno-associated viruses, retroviral vectors, and lentiviruses), which serve equivalent functions.

In certain aspects, a vector of the invention encodes an RNA silencing agent described supra, e.g., small hairpin RNAs (shRNAs). Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). supra; Miyagishi and Taira (2002), Nature Biotechnol., 20, 497-500; Paddi son et al. (2002), supra; Paul (2002), supra; Sui (2002) supra; Yu et al. (2002), supra. Such expression constructs may include one or more inducible promoters, RNA Pol III promoter systems such as U6 snRNA promoters or H1 RNA polymerase III promoters, or other promoters known in the art. The constructs can include one or both strands of the RNA silencing agent. Expression constructs expressing both strands can also include loop structures linking both strands, or each strand can be separately transcribed from separate promoters within the same construct. Each strand can also be transcribed from a separate expression construct, Tuschl (2002), supra.

### VI. Loading Payloads, e.g., Nucleic Acid Payloads into/onto Peptide-Conjugated Yeast Cell Wall Particles, e.g., Peptide-Conjugated Glucan Particles or Amine-Modified Glucan Particles

The present invention relates to a new, single-component siRNA delivery vehicle, which is particularly useful in a variety of in vitro and in vivo gene silencing applications. Preferred formulations have peptides (e.g., short, non-toxic, delivery peptides) covalently attached to the glucan particles or shells, which preferred formulations showing optimal siRNA binding to peptides and gene silencing in vitro and/or in vivo with minimal peptide length. In another aspect, preferred formulations have amines (e.g., short, non-toxic, small molecule amines) covalently attached to the glucan particles or shells, which preferred formulations showing optimal siRNA binding to amines and gene silencing in vitro and/or in vivo.

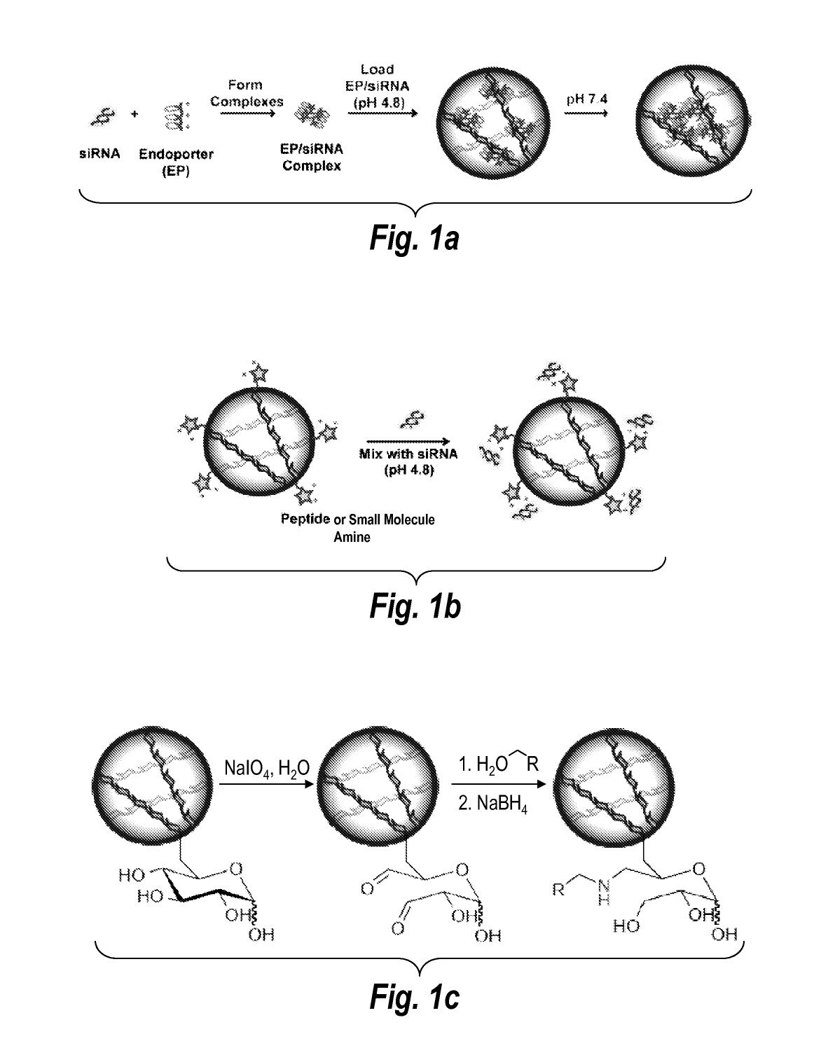

FIG. 1 depicts the strategy for simple loading of YCWPs. The loading strategy for previous formulation of particles (see e.g., Tesz et al.) involved (1) formation of siRNA/DP complexes, (2) siRNA/DP complex loading in a hydrodynamic volume into the glucan shells, and finally (3) entrapment of the complexes in the particles (FIG. 1(a)). By contrast, the new, single component particles of the invention can be loaded with siRNA simply by mixing siRNA with glucan particles previously conjugated with peptide, e.e., sDPs. Covalent attachment of a peptide to the glucan shell facilitates not only electrostatic binding of siRNA to this single component delivery system but also escape from the endosomes.

In exemplary embodiments, one or more sample(s) of prepared pcGPs or amPGs e.g., 1, 2, 5, 10, 20 mg pcGPs or amGPs are combined or incubated with 50, 100, 200 or 500 ug siRNA in an appropriate volume of solution (e.g., buffer) and incubation is allowed to proceed for example, for 10-30 minutes, 30 minutes-1 hour, 1-4 hours, 4-8 hours, 8-12 hours, 12-16 hours, 16-20 hours, 20-24 hours or more, to allow sufficient incorporation of siRNA into/onto (complexing with) pcGPs or amGPs. In an exemplary embodiment, pcGPs or amGPs are loaded with siRNA followed by addition of an appropriate amount of buffer (e.g., 100 uL buffer, 200 uL buffer, 500 uL buffer, 1 mL buffer) to give the desired final concentration of siRNA (2-5 nM, 5-50 nM, 50-100 nM).

Preparations of pcGPs and/or amGPs have advantages over the art-described technologies including, for example,
high degree of consistency when using simple, one component system “off the shelf”; e.g., consistency between multiple nucleic acid applications or drug delivery administrations;
lower cost of synthesis as efficient, large-batch synthesis of pcGPs is possible using GPs and peptides, e.g., delivery peptides; followed by small-scale preparation of individual siRNA-containing pcGPs

The delivery system described herein is improved over the art-described, multi-component systems made using the layer-by-layer (LBL) approach in that, particles made by the LBL approach retained a significant amount of siRNA within the GPs when administered in vivo, whereas the pcGPs and/or amGPs of the present invention allow for more efficient release of the nucleic acid, e.g., siRNA payload or cargo.

Likewise, the delivery system described herein is improved over the art-described systems in which siRNA are complexed with delivery peptides, e.g., Endoporter™ before loading into GPs.

There are several advantages of the new system over the previously described systems. For example, the delivery system of the invention is a simpler single-component formulation versus the system published in e.g., Tesz et al., which contains multiple components. A single-component formulation makes loading the particles with therapeutic cargo more facile and importantly, more reproducible and uniform. Loading of the particles of the invention entails a single step, simply mixing the particle with the therapeutic of interest. In contrast, loading of particles according to art-described methods involves multiple steps, as depicted in FIG. 1.

In addition, the delivery systems of the invention can be loaded with higher concentrations of siRNA. By contrast, the art-described systems are limited to a certain concentration of siRNA to avoid the presence of free siRNA/DP complexes. In using the particular peptides of the invention, the pcGPs and/or amGPs can be loaded with the concentration of siRNA desired for the intended purpose.

In addition, the lack of free siRNA/DP complexes eliminates possible non-specific knockdown in other non-phagocytic cell types. Free siRNA/DP nanoparticles are able to silence genes in virtually all cells, while pcGPs only enter phagocytes such as macrophages. Without being bound in theory, the same advantage is postulated when using amGPs.

The new system also features, in particular, the use of shorter peptide sequences (sDPs) and/or small molecule amines, thus avoiding the potential for an immunogenic response to a longer peptide.

Moreover, it was surprisingly found that, even when conjugated to GPs, the peptides, e.g., delivery peptides of the pcGPs were quite efficient in facilitating endosomal processing and siRNA payloads readily escaped the endosomal membranes of cells after administration. Contrary to what might be expected, conjugation of a peptide component of the peptide:siRNA complexes did not detrimentally trap or retain the siRNA payload and good in vivo delivery of siRNA payload was obtained.

Moreover, as described herein, amGPs have the further advantage of being suited to acidic environments, exhibiting desirable stability and release properties.

### VII. Methods of Use

Peptide-modified glucan particles (e.g., pcGPs) and/or amino-modified glucan particles, are a promising vector for the efficient delivery of therapeutic cargoes. In particular, we have demonstrated that these particles can be used to deliver siRNA in vivo in mice. Pharmaceutical and/or biotechnology companies can develop the technology to deliver siRNA targeting a protein of interest. The particles can be used to achieve siRNA therapy for a variety of diseases of clinical importance.

The modified GPs (e.g., pcGPs and/or amGPs) of the invention can be used in a variety of pharmaceutical applications, for example, in the delivery of therapeutics to treat a variety of diseases in patients, to deliver diagnostic agents e.g., for imaging, detecting and/or monitoring disease conditions such and the like. The modified GPs of the invention can be used to deliver the therapeutic or diagnostic agents to the appropriate target, e.g., a target tissue in vivo, when the agents can be released to carry out their intended function.

Using the delivery systems of the invention, efficient delivery of siRNA cargo was demonstrated in both cells in vitro, and in animal models. In particular, at least 80% knockdown of target mRNA was observed in proof-of-concept experiments in which siRNA was delivered to animals either via an intraperitoneal or intravenous route. Proof-of-concept and validation studies can also be made in animals in which siRNA-loaded pc-YCWP (pcGP) or siRNA-loaded am-YCWP (amGP) formulations are administered via an oral route of administration, taking full advantage of the specific ability of the YCWPs (GPs) to deliver cargo/payload to selected cells of the immune system.

Optimal pc-YCWP (e.g., pcGPs) and/or amGP formulations comprising therapeutically-active siRNAs can be developed for therapeutic administration, for example, in human subjects.

In exemplary embodiments, the pcGPs and/or amGPs of the invention are used in the therapeutic treatment of inflammatory diseases, as defined herein.

In preferred aspects, the amGPs of the invention are used in the therapeutic treatment of inflammatory bowel disease (IBD.) Without being bound in theory, the amGPs (and/or certain pcGPs) of the invention, are believed to protect and retain cargoes (e.g., siRNAs) in the acidic environment of the inflamed gut and release cargo, e.g., siRNA, into the cytoplasm of macrophages within the gut upon phagocytosis.

Inflammatory bowel diseases (IBDs) including colitis and Crohn's diseases impair health and quality of life of millions worldwide (Sartor R B (2006) Nat. Clin. Practice. Gastroenterol Hepatol. 3:390-407. IBD is characterized by a chronic inflammation of the digestive tract associated with increased expression of inflammatory genes such as tumor necrosis factor α (TNF-α). Poorly controlled IBD significantly increases the rate and mortality of colon cancer (Triantafillidis et al (2009) Anticancer Res. 29:2727-2737. Current therapies for IBD include TNF-α blockers administered systemically. However these drugs are associated with a high risk of infection, due to systemic loss of immune function (Neilsen et al. (2013) NEJM 369:754-762; Emi Aikawa et al. (2010) Clin. Rev. Allergy & Immunol. 38:82-89. Therefore, a safe orally delivered therapeutic that specifically decreases TNF-α in the gut with minimal systemic exposure would represent a major advantage in the treatment of IBD.

Previous studies, using various delivery systems, have demonstrated that orally administered siRNA targeting TNF-α improved the symptoms of chemically induced IBD in mice (Wilson et al. (2010) Nat. Materials 9:923-928; Kriegel et al. (2011) J. Controlled Release 150:77-86). However, these approaches lack the ability to target specific cell types, such as macrophages, which are essential for the development of IBD. Therefore, an oral siRNA delivery system that specifically decreases TNF-α in macrophages in the gut would represent a major advantage in the treatment of IBD.

Preliminary data indicated that exemplary amGP formulations, of the invention, in particular, ethylenedi amine-modified GP, readily bind the siRNA in acidic pH. When administered orally this formulation significantly reduced TNF-α expression in the distal colon of mice with colitis, without affecting circulating TNF-α levels in serum. In addition, silencing with this new formulation reduced the weight loss associated with colitis induced by dextran sulfate salt (DSS). These data demonstrate the efficacy of the amGPs of the invention in treating, for example, IBD. Such treatment is particularly suited as a therapeutic strategy to safely, specifically and efficiently deliver RNAi therapeutics via oral route to silence gene expression in the inflamed gut with minimal systemic exposure and ameliorate the symptoms in TBD. The success of this study will present significant therapeutic potential for the treatment of IBD.

Routes of administration include but are not limited to oral; other contemplated routes of administration include buccal, sublingual, pulmonary, transdermal, transmucosal, as well as subcutaneous, intraperitoneal, intravenous, and intramuscular injection. Local administration is an exemplary delivery route. Preferred routes of administration are oral, intravenous and local administration.

The particulate delivery system of the present invention is administered to a patient in a therapeutically effective amount.

The particulate delivery system can be administered alone or as part of a pharmaceutically acceptable composition. In addition, a compound or composition can be administered all at once, as for example, by a bolus injection, single oral dose, or the like, or can be administered multiple times, such as by a series of injections or oral doses, delivered substantially uniformly over a period of time. It is also noted that the dose of the compound can be varied over time. The particulate delivery system can be administered using an immediate release formulation, a controlled release formulation, or combinations thereof. The term “controlled release” includes sustained release, delayed release, and combinations thereof.

In exemplary embodiments featuring delivery of siRNA, a therapeutically effective amount is achieved by more than one administration of siRNA:pcGPs and/or amGPs of the invention. For example, siRNA:pcGPs and/or pcGPs can be administered daily for a period of time, e.g., over several days, to achieve a cumulative dose of siRNA. Administration can be daily, every other day, weekly, bi-weekly, monthly, etc., until the preferred cumulative dose is achieved. In proof-of concept experiments described infra, a cumulative dose of 10 nmol siRNA in 1 mg pcGPs was achieved using 5 daily doses. This corresponds to a total dose of 4.3 mg of siRNA/kg of body weight and 28.6 mg of shells/kg of body weight. Exemplary dosing in mice can include, for example, 0.1-1 mg siRNA, 1-5 mg siRNA, 5-10 mg siRNA, 10-100 mg siRNA, etc. and 1-10 mg pcGPs, 10-20 mg pcGPs, 10-50 mg pcGPs, 10-100 mg pcGPs, 50-100 mg pcGPs, 100-200 mg pcGPs, 200-500 mg pcGPs, etc. per kg of body weight. In proof-of concept experiments described infra, a cumulative dose of 10 nmol siRNA in 2 mg amGPs was achieved using 5 daily doses. This corresponds to a total dose of 4.3 mg of siRNA/kg of body weight and 57.2 mg of shells/kg of body weight. Exemplary dosing in mice can include, for example, 0.1-1 mg siRNA, 1-5 mg siRNA, 5-10 mg siRNA, 10-100 mg siRNA, etc. and 1-10 mg amGPs, 10-20 mg amGPs, 10-50 mg amGPs, 10-100 mg amGPs, 50-100 mg amGPs, 100-200 mg amGPs, 200-500 mg amGPs, etc. per kg of body weight. These amounts/concentrations can be varied (e.g., lower or higher concentrations of each component can be used) depending on the application. In addition, the frequency and number of treatments can be varied. Based on exemplary dosing in animal models, the skilled artisan can determine appropriate dosing regimes for use in human therapeutics.

A pharmaceutical composition of the invention can be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

In addition, a particulate delivery system of the present invention can be administered alone, in combination with a particulate delivery system with a different agent or compound, e.g., with other pharmaceutically active compounds. The other pharmaceutically active compounds can be selected to treat the same condition as the particulate delivery system or a different condition.

Another aspect of the invention relates to a kit comprising a composition of the invention and instructional material. Instructional material includes a publication, a recording, a diagram, or any other medium of expression which is used to communicate the usefulness of the composition of the invention for one of the purposes set forth herein in a human. The instructional material can also, for example, describe an appropriate dose of the composition of the invention. The instructional material of the kit of the invention can, for example, be affixed to a container which contains a composition of the invention or be shipped together with a container which contains the composition. Alternatively, the instructional material can be shipped separately from the container with the intention that the instructional material and the composition be used cooperatively by the recipient.

In some embodiments, a kit may comprise a first composition of the invention comprising a particulate delivery system, for example, in a pharmaceutically acceptable carrier; and a second composition which is a pharmaceutically active compound or diagnostic agent, optionally in a pharmaceutically acceptable carrier. The kit can comprise a container for the separate compositions, such as a divided bottle or a divided foil packet. Additional examples of containers include syringes, boxes, bags, and the like. Typically, a kit comprises directions for the formulation and/or administration of the separate components. Kits containing more than one pharmaceutically active agent, optionally in particulate delivery systems of the invention, are also contemplated and such forms are particularly advantageous when the separate components are preferably administered in different dosage forms (e.g., oral and parenteral), are administered at different dosage intervals, or when titration of the individual components of the combination is desired by the prescribing physician.

A particulate delivery system composition, optionally comprising other pharmaceutically active compounds, can be administered to a patient either orally, rectally, parenterally, (for example, intravenously, intramuscularly, or subcutaneously) intracistemally, intravaginally, intraperitoneally, intravesically, locally (for example, powders, ointments or drops), or as a buccal or nasal spray.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intraarterial, intramuscular, or intrasternal injection and intravenous, intraarterial, or kidney dialytic infusion techniques.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parenterally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

Solid dosage forms for oral administration include capsules, tablets, powders, and granules. In such solid dosage forms, the particulate delivery system is optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Oral compositions can be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the particulate delivery system to a specific location within the gastrointestinal tract. Such systems permit delivery at a predetermined time and can be used to deliver the particulate delivery system, optionally together with other additives that may alter the local microenvironment to promote stability and uptake, directly without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, almond oil, arachis oil, coconut oil, cottonseed oil, groundnut oil, corn germ oil, olive oil, castor oil, sesame seed oil, MIGLYOL®, glycerol, fractionated vegetable oils, mineral oils such as liquid paraffin, tetrahydrofurfuryl alcohol, polyethylene glycols, fatty acid esters of sorbitan, or mixtures of these substances, and the like. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances, and the like. Liquid formulations of a pharmaceutical composition of the invention that are suitable for oral administration can be prepared, packaged, and sold either in liquid form or in the form of a dry product intended for reconstitution with water or another suitable vehicle prior to use.

A pharmaceutical composition of the invention can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the particulate delivery system suspended in a low-boiling propellant in a sealed container. Dry powder compositions may include a solid fine powder diluent such as sugar and are conveniently provided in a unit dose form. Low boiling propellants generally include liquid propellants having a boiling point below 65 degrees F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent (preferably having a particle size of the same order as particles comprising the particulate delivery system).

Pharmaceutical compositions of the invention formulated for pulmonary delivery can also provide the active ingredient in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface active agent, or a preservative such as methylhydroxybenzoate.

The formulations described herein as being useful for pulmonary delivery are also useful for intranasal delivery of a pharmaceutical composition of the invention. Another formulation suitable for intranasal administration is a coarse powder comprising the particulate delivery system. Such a formulation is administered in the manner in which snuff is taken i.e. by rapid inhalation through the nasal passage from a container of the powder held close to the nares.

A pharmaceutical composition of the invention can be prepared, packaged, or sold in a formulation suitable for buccal administration. Such formulations can, for example, be in the form of tablets or lozenges made using conventional methods, and can, for example, comprise 0.1 to 20% (w/w) particulate delivery system, the balance comprising an orally dissolvable or degradable composition and, optionally, one or more of the additional ingredients described herein. Alternately, formulations suitable for buccal administration can comprise a powder or an aerosolized or atomized solution or suspension comprising the particulate delivery system.

The following examples illustrate the preparation of certain specific compounds according to the present technology. A skilled artisan appreciates that the invention is not limited to the exemplary work described or to the specific details set forth in the examples.

A skilled artisan further appreciates that the experimental conditions depicted in the following examples can be varied by as much as 2%, 5%, 10% or 20% above or below the listed amount, temperature, concentration, pH, time and rpm in order to optimize the conditions to achieve the desired results from the experiments.

### EXAMPLES

RNA interference (RNAi) is a biological mechanism wherein double-stranded RNAs (siRNAs) can be used to reduce expression of target proteins, and has emerged as a promising therapeutic strategy for the treatment of many diseases, including inflammatory diseases and malignancies. However, development of clinical applications of RNAi therapy has been hindered by the lack of clinically suitable, safe, and effective delivery vehicles. Macrophages are particularly attractive targets for RNA interference therapy because they promote pathogenic inflammatory responses in diseases such as rheumatoid arthritis, atherosclerosis, and diabetes. Thus, siRNA delivery systems capable of targeting immune cells present a promising therapeutic approach for the treatment of these and other major human diseases.

The data presented herein demonstrate the synthesis and evaluation of a microparticulate technology capable of delivering small interfering RNA (siRNA) to immune cells (e.g., macrophages). In particular, the data presented herein demonstrate the synthesis and evaluation of peptide-modified glucan particles and/or amine-modified glucan particles for the delivery of therapeutic siRNA. This work represents a significant advance over technologies described in the art that employ siRNA delivery systems based on glucan microparticles derived from baker's yeast. For example, it had been previously demonstrated by the instant inventor(s) that β-1,3-D-glucan-encapsulated siRNA particles (GeRPs) could potently silence genes in mouse macrophages in vitro and in vivo. A major advance is to simplify the prior art technology which featured a complex, multi-component system.

The present invention now features simple, single-component systems that, when mixed with siRNA, form very efficient delivery vehicles. In order to create an exemplary new single-component system, an amphipathic peptide was covalently attached to the glucan shells using reductive amination chemistry. Modification of the glucan particles (GPs) facilitates the easy preparation of siRNA-loaded particles. The new chemically-modified glucan particles (GPs) can efficiently deliver siRNA to macrophages in vivo with minimal toxicity. The data presented herein show that peptide-modified glucan particles consisting of a 14-amino acid peptide conjugated to the glucan particle (pcGP-14s) were able to efficiently deliver siRNA to peritoneal macrophages in healthy mice following intraperitoneal (i.p.) injection. The data generated using this simplified formulation demonstrates a useful technology for the delivery of therapeutic siRNAs. The technology has particular potential for use in the treatment of inflammation-related diseases.

To demonstrate the utility of the pcGP-14s for delivering siRNA to macrophages in vivo, e.g., for the treatment of inflammatory diseases, we studied the efficacy of the pcGP-14s in a model of inflammation, obesity. Inflammation of the adipose tissue has been suggested as a key link between obesity and insulin resistance leading to type 2 diabetes in humans and animals. These data show that i.p. injections of pcGP-14s_loaded with an siRNA sequence against an inflammatory cytokine, osteopontin, reduced its expression by about 80% in the adipose tissue macrophages of obese insulin-resistant mice. Importantly, reducing inflammation with pcGP-14s resulted in an improved metabolic phenotype. The data generated with this simplified GP formulation demonstrates a useful technology for the delivery of therapeutic siRNAs.

To demonstrate the utility of amGPs for delivering siRNA to macrophages in vivo, e.g., for the treatment of inflammatory diseases, we studied the efficacy of amGPs in a model of inflammation, IBD. Oral delivery of amGPs loaded with an siRNA sequence against an inflammatory cytokine, TNF-α, significantly reduced its expression in colon. Importantly, reducing inflammatory cytokine expression in this animal model correlated with reduction in body weigh loss. The data generated with this simplified GP formulation demonstrates a useful technology for the delivery of therapeutic siRNAs.

### Example 1

### Preparation of Glucan Particles (GPs) (or Glucan Shells, GS)

β-1,3-D-glucan particles were prepared using art-described methodologies (see e.g., Biochem J and PNAS, infra.). Briefly, β-1,3-D-glucan particles were prepared by suspending Saccharomyces cerevisiae (100 g of SAF-Mannan; SAF Agri, Milwaukee, Wis.) in 1 L of 0.5 M NaOH and heating to 80° C. for 1 h. The insoluble material containing the yeast cell walls was collected by centrifugation at 10000 rpm for 10 min. This insoluble material was then suspended in 1 L of 0.5 M NaOH, and incubated at 80° C. for 1 h. The insoluble residue was again collected by centrifugation (10000 rpm for 10 min) and washed five times with 1 L of water, three times with 1 L of propan-2-ol, and three times with 1 L of acetone. The resulting slurry was placed in a glass tray and dried at room temperature (20° C.) to produce 8.1 g of a fine, slightly off-white powder.

### Example 2

### Exemplary Peptides

All peptides were purchased from 21^{st} Century Biochemicals (Marlborough, Mass.).

### Peptide Nomenclature

For the purposes of the working Examples, short delivery peptides can be referred to herein as “sDP(number of amino acids in sequence)”. For example, a truncated version of a peptide disclosed in U.S. Pat. No. 7,084,248 consisting of 14 amino acids would be named sDP(14). When conjugated to GPs/GSs, sDPs can also be referred to using abbreviated nomenclature referencing peptide length only, e.g., pcGP-14.

### Exemplary Peptide Sequences Include

(SEQ ID NO: 39)

sDP(14)-H₂N-LHLLHHLLHHLHHL-CONH₂

(SEQ ID NO: 45)

sDP(10)-H₂N-LHLLHHLLHH-CONH₂

(SEQ ID NO: 48)

sDP(5)-H₂N-LHHLL-CONH₂

### Example 3

### Synthesis of Peptide-Conjugated Glucan Particles or Shells

All peptide-conjugated GPs were prepared in a similar fashion. Peptides were covalently attached to the GP via the N-terminal amine using reductive amination chemistry. For ease of nomenclature, the peptide-modified particles are named pcGP followed by the number of amino acids in the peptide sequence conjugated thereto. For example a GP modified with a peptide consisting of 14 amino acids would be named pcGP-14. Details for the synthesis of pcGP-14 are provided as an example. As described herein, the terms GP and GS can be used interchangeable.

### Partial Oxidation of GPs/GSs

GPs/GSs (100 mg) were resuspended in 7 mL of dd-H₂O by sonication. Sodium periodate (22 mg, 0.103 mmol) was dissolved in 3 mL of dd-H₂O and this solution was added to the solution containing GPs/GSs. The mixture was stirred for 24 h at 37° C. Oxidized GPs/GSs were isolated by centrifugation at 3800 rpm for 10 min, and the resulting pellet was washed with dd-H₂O (2×10 mL) by resuspension followed by centrifugation and removal of the supernatant. The samples were used immediately for peptide modification.

Synthesis of sDP(14)-GS (or pcGP-14)—Reductive Amination

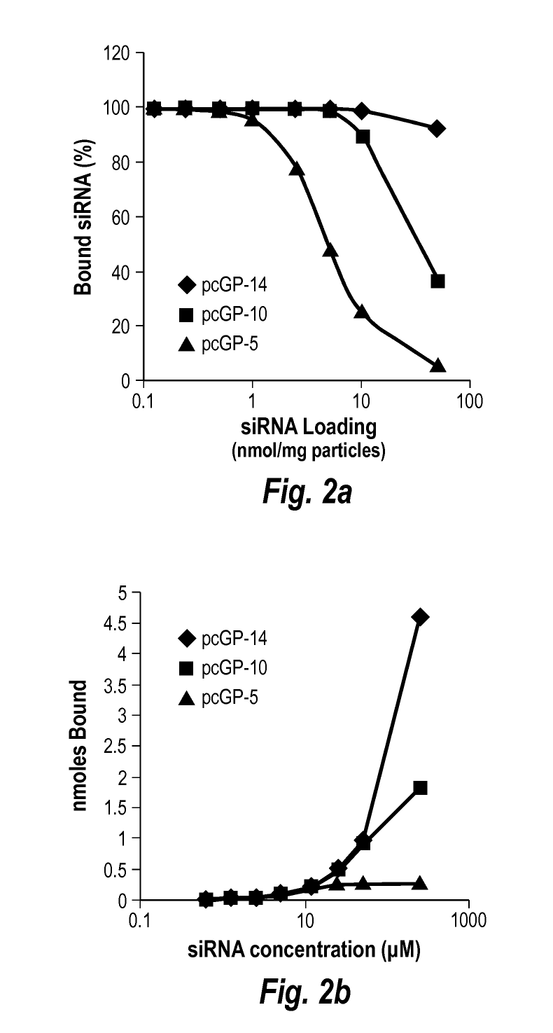

Oxidized GPs/GSs were resuspended in 8 mL of dd-H₂O by sonication. Borate buffer (2 mL, 0.1 M, pH 9.5) and sDP(14) (333 mg/mL in DMSO) was added to the GS suspension and mixed overnight at 37° C. The reduction was performed for 72 h at room temperature by adding sodium borohydride (14 mg) to the GS solution. sDP (14)-GS were isolated by centrifugation at 3800 rpm for 10 min and washed thoroughly with dd-H₂O (8×30 mL) and ethanol (2×30 mL) by resuspension followed by centrifugation and removal of the supernatant. The sDP (14)-GS were then flash-frozen and residual water was removed by lyophilization. FIG. 2 depicts the covalent coupling of peptide(R) amines to glucan particles via reductive amination. sDP (14)-GS is referred to herein (interchangeably) as sDP (14)-GP or as pcGP-14.

### Alternative Synthesis of Peptide-GS

A second method uses epibromohydrin to activate the particles followed by amination.

GS were resuspended in dd-H₂O and 1 M NaOH. Epibromohydrin was added to form a 10% epibromohydrin solution. The reaction proceeded overnight at 37° C. The activated GS were washed thoroughly and amine was added. The reaction was allowed to proceed overnight at 37° C. under gentle agitation. The amine-modified GS were isolated by centrifugation and washed thoroughly.

### Preparation of Fluorescently-Labeled GS

Fluorescently-labeled sDP (14)-GS (or pcGP-14) were prepared in the same manner as described above, except that either 5-(((2-(Carbohydrazino) methyl)thio)acetyl)Aminofluorescein (Invitrogen, 1 mg/mL in dd-H₂O) or Cascade Blue hydrazide (Invitrogen, 1 mg/mL in dd-H₂O) was added to the sDP (14) solution.

### Example 4

### Loading of Peptide-Conjugated Glucan Particles or Shells and Characterization of Peptide-Conjugated GPs/GSs

Method for Loading sDP(14)-GS (or pcGP-14) with siRNA

To load siRNA in sDP (14)-GS (pcGP-14), siRNA (Dharmacon) was mixed with sDP(14)-GS in sodium acetate buffer (30 mM, pH 4.8) and incubated for 20 minutes at room temperature. The siRNA-loaded particles were then diluted with PBS to obtain a final particle concentration of 1 mg/mL and sonicated [15 s at 18 W at room temperature using a Sonicator 3000 (Misonix)] to ensure homogeneity of the particle preparation. Particles were aliquoted into tubes for daily dosing and either flash-frozen in liquid nitrogen and stored at −20° C., or kept at 4° C.

### Quantification of Peptide Loading

Peptide quantification was performed using a BCA Protein Assay Kit (Pierce Biotechnology, Rockford, Ill.). The peptide-modified particles (10 mg) were incubated in DMSO (1 mL) at 50° C. for 2 h to dissolve the particles. The samples were centrifuged to remove insoluble material and diluted with DPBS. The solution was then analyzed for the concentration of peptide using the kit according to the manufacturer's instructions. A standard curve was prepared using known concentrations of peptide.

FIG. 1b depicts the strategy for pcGP or amGP formulation (as compared to art-described formulations, FIG. 1a). The new single component particles can be loaded with siRNA simply by mixing siRNA with glucan particles previously conjugated with peptide (pcGPs) or with small molecule amines. Covalent attachment of a peptide to the glucan shell facilitates not only electrostatic binding of siRNA to this single component delivery system but also escape from the endosomes.

siRNA Binding Assay

The ability of peptide-modified GPs to electrostatically interact with siRNA was evaluated in PBS (pH 7.4) or sodium acetate buffer (30 mM, pH 4.8). Peptide-modified glucan shells (10 mg/mL in either PBS (pH 7.4) or acetate buffer (30 mM, pH 4.8)) were loaded with different concentrations of fluorescently-labeled siRNA as described above. Briefly, Peptide-modified GPs (10 mg/mL in either PBS or acetate buffer) were loaded with different concentrations of fluorescently-labeled siRNA (Dy547-siRNA, Dharmacon, Pittsburgh, Pa.) ranging from 1.25-250 μM by mixing siRNA with peptide-modified GPs and incubating for 20 min at room temperature. Particles loaded with siRNA were sedimented by centrifugation at 9000 rpm for 5 min and the supernatant was assessed for siRNA content by measuring the fluorescence on a microplate reader. Free siRNA was calculated relative to the siRNA control that did not contain particles (siRNA alone, 100% free, 0% bound).

Results

The glucan particles (GPs) were prepared by treating baker's yeast with a series of chemical extractions to yield hollow, porous shells that are approximately 2-4 μm in diameter (Tesz, Biochem. J.). Peptides were covalently attached to the GPs via the N-terminal amine using reductive amination chemistry (FIG. 1c). The glucan particles were first lightly oxidized with sodium periodate. Amine modification was then performed between the peptide and the oxidized glucan particles using sodium borohydride as the reducing agent. A small library of peptide-modified GPs containing amphipathic peptide sequences (based on truncations of the EP sequence, see Table 2) was prepared to study the effect of varying the peptide length on siRNA delivery efficiency. For ease of nomenclature, the peptide-modified particles are named pcGP followed by the number of amino acids in the sequence. For example, a particle modified with a peptide consisting of 14 amino acids would be named pcGP-14. Peptide incorporation was quantified using a bicinchoninic acid (BCA) assay. Using this method, the peptide content in the particles was found to be approximately 0.2 μmol of peptide per mg of GP.

TABLE 2
Selected Peptide Modifications

H₂N-LHHLLHHLLHHLHHLLHHLHHLLHHL-CONHCH₃
SEQ ID NO: 49
H₂N-LHLLHHLLHHLHHL-CONH₂ (SEQ ID NO: 39)
H₂N-LHLLHHLLHH-CONH₂ (SEQ ID NO: 45)
H₂N-LHHLL-CONH₂ (SEQ ID NO: 48)

To determine the ability of the peptide-modified GPs to electrostatically interact with siRNA, fluorescently-labeled siRNA was incubated with the modified particles at different concentrations and at different pH conditions. As predicted, the different peptide-modified particles bind siRNA with varying degrees of strength (FIGS. 2a and 2b). In addition, the pH conditions altered the ability of the particles to bind siRNA. Acidic pH increases the positive charges on the histidine residues of the peptides leading to stronger binding to siRNA under these conditions. The peptide-modified GPs that showed the highest siRNA binding ability, the pcGP-14s, were selected for further studies.

### Example 5

### In Vitro Transfection Experiments

### Peritoneal Macrophage Preparation

All mice were purchased from Jackson Laboratory. Mice were housed on a 12-h light/dark schedule and had free access to water and food. All procedures involving animals were approved by the Institutional Animal Care and Use Committee at the University of Massachusetts Medical School.

C57BL6/J male mice (10 weeks old) were i.p. (intraperitoneally) injected with 4% thioglycollate broth (Sigma-Aldrich). At 5 days following injection, mice were sacrificed and the peritoneal cavity was washed with 5 mL of ice-cold PBS to isolate PECs (peritoneal exudate cells). Peritoneal fluid was filtered through a 70 μM diameter pore nylon mesh and centrifuged at 270 g for 10 min. The pellet was first treated with red blood cell lysis buffer (8.3 g of NH₄Cl, 1.0 g of KHCO₃ and 0.037 g of EDTA dissolved in 1 L of water) and then plated in DMEM (Dulbecco's modified Eagle's medium) supplemented with 10% (v/v) FBS (fetal bovine serum), 50 μg/ml streptomycin and 50 units/ml penicillin. Cells were plated at a density of 1 million cells/well in 6-well plates. At 24 h after isolation, PECs were treated with siRNA.

### Peptide-siRNA In Vitro Treatment

For in vitro treatment, siRNA was incubated with peptide (1:25 molar ratio of siRNA to peptide) for 30 minutes at room temperature to allow for complexes to form. Peptide-siRNA complexes were diluted to the appropriate concentration in complete media and added to peritoneal exudate cells (100 pmol siRNA/well). The cells were allowed to grow for an additional 72 h before being analyzed for gene expression.

### Example 6

### In Vivo Transfection Experiments

Preparation of pcGP-14s

For in vivo treatment, pcGP-14s were loaded with siRNA (Dharmacon; sequences listed in Table 3) targeting either F4/80, osteopontin (OPN), or a scrambled (Scr) control sequence.

TABLE 3

| Target | siRNA sequence |
|---|---|
| Scr | 5′-CAGUCGCGUUUGCGACUGG-3′ SEQ ID NO: 50 |
| F4/80 | 5′-GUGAAUGAGUGUCAAGAUA-3′ SEQ ID NO: 51 |
| OPN | 5′-CCACAUGGCUGGUGCCUGA-3′ SEQ ID NO: 52 |

To load siRNA in pcGP-14s, siRNA (10 nmoles) was mixed with pcGP-14s (1 mg) in sodium acetate buffer (30 mM, pH 4.8) and incubated for 20 min at room temperature. The siRNA-loaded particles were then diluted with PBS to obtain a final particle concentration of 1 mg/mL and sonicated [15 s at 18 W at room temperature using a Sonicator 3000 (Misonix)] to ensure homogeneity of the particle preparation. Particles were aliquoted into tubes for daily dosing and either flash-frozen in liquid nitrogen and stored at −20° C., or kept at 4° C.

Preparation of Control siRNA-Loaded GeRPs

siRNA-loaded GeRPs were prepared according to art-described methods, see e.g., (Tesz et al. Biochem. J., 436: 351-362 (2011); and Aoudi et al., PNAS 110: 8278-8283 (2013)). Briefly, to load siRNA in unmodified glucan shells, 3 nmoles siRNA (Dharmacon, Pittsburgh, Pa.) were incubated with 50 nmoles Endo-Porter (EP, Gene Tools, Philomath, Oreg.) in 30 mM sodium acetate pH 4.8 for 15 min at room temperature in a final volume of 20 μL. The siRNA/EP solution was added to 1 mg of glucan shells and then vortexed and incubated for 1 h. The siRNA-loaded GPs, or GeRPs, were then resuspended in PBS to obtain a final particle concentration of 1 mg/mL and sonicated to ensure homogeneity of the GeRP preparation. GeRPs were kept at 4° C.

### Particle Administration for F4/80 Knockdown

C57BL6/J male mice (8 weeks old) were i.p. injected once a day for 5 days with 1 mg of sDP (14)-GS (pcGP-14) containing 10 nmoles of siRNA. On day 6, mice were sacrificed and the peritoneal cavity was washed with 5 mL of ice-cold PBS to isolate peritoneal exudate cells (PECs). Peritoneal fluid was filtered through a 70 μm pore nylon mesh and centrifuged at 1200 rpm for 10 min. The pellet was first treated with red blood cell lysis buffer (8.3 g of NH₄Cl, 1.0 g of KHCO₃ and 0.037 g of EDTA dissolved in 1 L of water), and then plated in 6-well plates for 2-3 h in medium (Dulbecco's Modified Eagle's Medium (DMEM) supplemented with 10% (v/v) fetal bovine serum (FBS), 100 μg/ml streptomycin and 100 units/ml penicillin). The media was changed and the cells were incubated overnight to enrich for macrophages. Adherent cells were used for real-time PCR.

Results

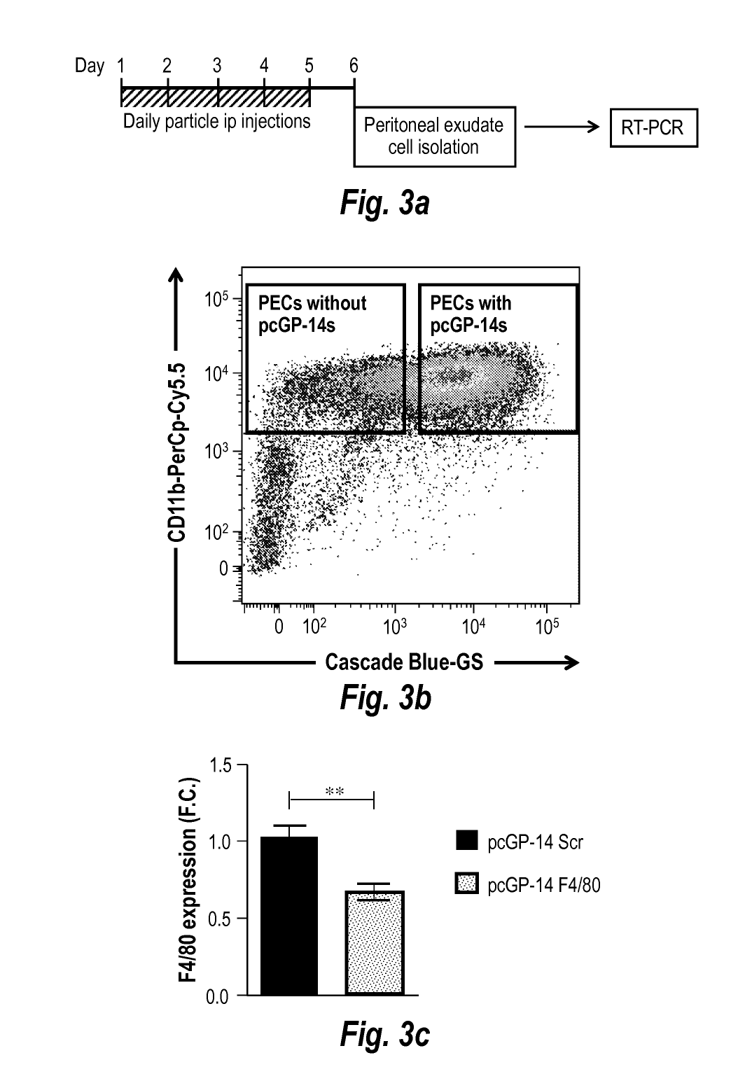

The ability of peptide-modified GPs to induce target-specific gene silencing was determined by treating mice with pcGP-14s loaded with either Scr or F4/80 siRNA following the protocol outlined in FIG. 3a. The data in FIG. 3 demonstrate that siRNA-loaded, peptide-conjugated glucan shells attenuate F4/80 mRNA expression in PECs. Mice were treated with PBS or sDP (14)-GS (pcGP-14) loaded with control, scrambled (Scr) or F4/80 siRNA the expression of F4/80 and other genes of interest was measured by real-time PCR in PECs. Statistically significant knockdown was observed in PECs treated with F4/80 siRNA-loaded sDP (14)-GS. Briefly, gene knockdown in peritoneal macrophages recovered from pcGP-14 treated mice is in the range of 30-50%, a modest but significant gene silencing (FIG. 3c). Other surface markers, such as CD68 and CD11c, were unchanged in PECs from mice treated with pcGP-14s loaded with F4/80 siRNA compared with pcGP-14s loaded with Scr siRNA (data not shown).

The exemplary F4/80 siRNA sequence is as follows:

(SEQ ID NO: 51)

5′-GUGAAUGAGUGUCAAGAUA-3′

### Phagocytosis by Marcophages Following Particle Administration

To test whether pcGP-14s can efficiently deliver siRNA to macrophages in vivo, normal mice were treated with PBS or particles loaded either with control scrambled (Scr) or F4/80 siRNA. Briefly, pcGP-14s were administered to C57BL6/J male mice via intraperitoneal (i.p.) injection once daily for five consecutive days (FIG. 3a). The day after the last injection, peritoneal exudate cells (PECs) were isolated and plated overnight to enrich for macrophages. PECs from mice treated with fluorescently-labeled particles (Cascade Blue) were stained with an antibody against the macrophage marker, CD11b, and the ability of macrophages to phagocytose the particles was analyzed by flow cytometry (FIG. 3b). FIG. 3b shows a representative dot plot of PECs from Cascade Blue pcGP-14 treated mice. FACS analysis showed that approximately 40-60% of the cells contained pcGP-14s and most of these cells were CD11b-positive (FIG. 3b, upper right gate). These results proved that pcGP-14s were taken up by macrophages in vivo in lean, healthy mice.

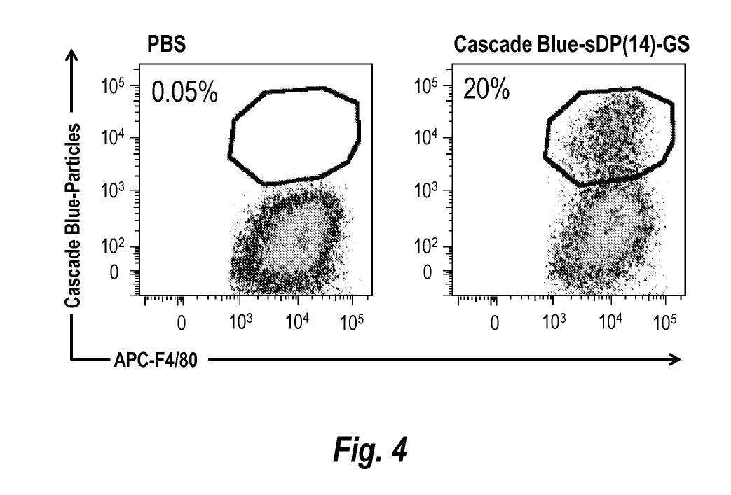

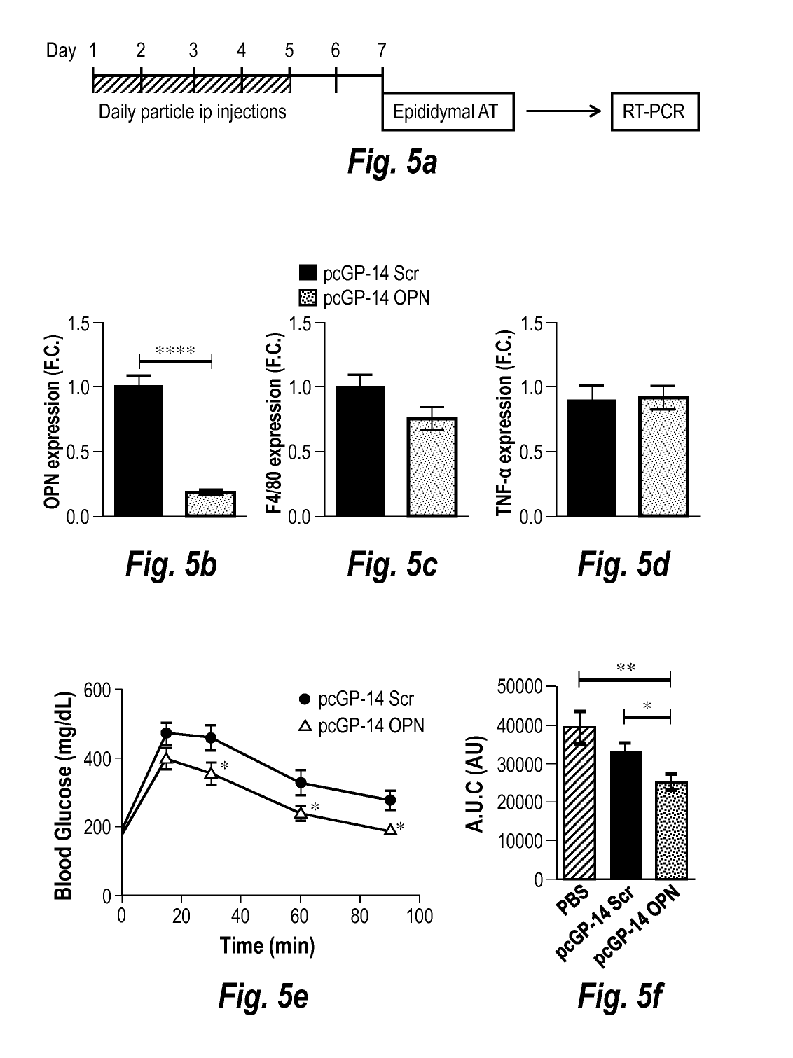

In separate experiments, peptide-conjugated glucan shells were found to undergo phagocytosis by macrophages in the epididymal adipose tissue (AT) of obese mice. FIG. 4 depicts FACS analysis showing adipose tissue (AT) macrophages containing peptide-conjugated particles. 5-week old ob/ob mice were injected once a day for 5 days with PBS or 1 mg of fluorescently-labeled sDP(14)-GS (pcGP-14) loaded with 10 nmoles of Scr siRNA. On day 7, mice were sacrificed and stromal vascular fraction (SVF) cells from the epididymal adipose tissue (AT) were isolated, stained, and analyzed by flow cytometry (FIG. 5a). The macrophage population was defined as F4/80⁺/CD11b⁺/Gr-1⁻ (FIG. 4).

Isolation of Macrophages from Adipose Tissue SVF

Adipose tissue SVF cells were prepared from collagenase-digested adipose tissue, as described previously (Aouadi et al., 2013). Briefly, epididymal fat pads were mechanically dissociated using the gentleMACS Dissociator (Miltenyi Biotec) and digested with collagenase at 37° C. for 45 minutes in Hank's buffered saline solution (HBSS) (Gibco, Life Technologies) containing 2% bovine serum albumin (American Bioanalytical) and 2 mg/ml collagenase (collagenase from clostridium histolyticum, Sigma). Samples were then filtered through 100 vim BD falcon cell strainers and spun at 300 g for 10 minutes at room temperature. The adipocyte layer and the supernatant were aspirated and the pelleted cells were collected as the stromal vascular fraction (SVF). The cells were then treated with red blood cell (RBC) lysis buffer, washed in PBS and then stained for flow cytometry.

### Flow Cytometry

For flow cytometry experiments, C57BL6/J male mice (8 weeks old) were treated as described above for WT mice with fluorescently-labeled pcGP-14s loaded with siRNA. On day 6, PECs were isolated and then plated in 10 cm tissue culture dishes overnight. At 24 hours after isolation, PECs were washed, scraped in PBS, and centrifuged at 1200 rpm for 10 min.

SVF cells (or PECs, as described above) from mice treated with Cascade-Blue sDP(14)-GS, were resuspended in PBS containing 1% BSA (FACS buffer) containing Fc block (clone 2.4G2, eBioscience, San Diego, Calif., USA) and allowed to block non-specific binding for 15 minutes at 4° C. Cells were then counted and incubated for an additional 20 minutes in the dark at 4° C. with fluorophore-conjugated primary antibodies or isotype control antibodies. Antibodies used in these studies included: F4/80-APC (clone C1:A3-1, AbD Serotec, Raleigh, N.C., USA), CD11b-PerCP-Cy5.5 (clone M1/70), Gr-1-APC-Cy7 (clone RB6-8C5), and Siglec-f-PE (clone E50-2440). Sample data were acquired on a BD LSRIT (BD Biosciences) and analyzed with FlowJo software (Tree Star). Sample data were initially gated on forward and side scatter, followed by a singlet cell gate, and then a gate to remove auto-fluorescent debris.

### Example 7

### Gene Silencing in Obesity, a Model of Inflammation

Obesity leads to a chronic, low-grade tissue inflammatory state, which may be a key pathogenic link between obesity and its metabolic sequelae. Thus, obesity was chosen as a model of inflammatory disease to test the ability of the pcGP-14s to deliver functional siRNA to macrophages in inflamed tissues in vivo. Adipose tissue inflammation is characterized by increased levels of inflammatory cytokines and chemokines that can exacerbate insulin resistance. Osteopontin (OPN) is a pro-inflammatory cytokine that is strongly upregulated in the obese adipose tissue of both mice and humans. In addition, OPN has been shown to play an important role in promoting inflammation and the accumulation of macrophages in the adipose tissue during obesity (Nomiyama, et al. Clin. Invest. 117, 2877-2888 (2007); Kiefer, Diabetes 59, 935-946 (2010).).

### Particle Administration for OPN Knockdown

Weight and fasting glucose tolerance test (GTT) were used to randomize the mice into different treatment groups. Genetically obese B6.V-Lep^{ob}/J (ob/ob) male mice (5 weeks old) were i.p. injected once a day for 5 days with 1 mg of sDP (14)-GS (pcGP-14) containing 10 nmoles of siRNA. On day 6, mice were fasted overnight. On day 7, glucose tolerance tests (GTT) were performed. Following the GTT, mice were sacrificed and various tissues were isolated for further analysis. Samples for RNA extraction were frozen in liquid nitrogen and stored at −80° C.

### Metabolic Studies

Glucose tolerance tests (GTTs) were performed on ob/ob animals following pcGP-14 treatment. Glucose (1 g/kg) was administered by i.p. injection. Blood samples were withdrawn from the tail vein at the indicated time, and glycemia was determined using glucometers (Alpha-Trak).

### Isolation of RNA and Real-Time PCR

RNA isolation was performed according to the TRIzol® Reagent protocol (Invitrogen, Grand Island, N.Y.). Tissues were homogenized using the gentleMACS Dissociator (Miltenyi Biotec) in TRIpure Isolation reagent (Roche Applied Science, Indianapolis, Ind., USA), and total RNA was isolated according to the manufacturer's instructions. cDNA was synthesized from 0.5-1 μg of total RNA using the iScript cDNA Synthesis Kit (Bio-Rad, Hercules, Calif.) according to the manufacturer's instructions. For real-time PCR, synthesized cDNA, forward and reverse primers, along with SYBR Green were run on the CFX96 Realtime PCR System (Bio-Rad). The ribosomal mRNA 36B4 was used as an internal loading control, as its expression did not change over a 24 h period with the addition of siRNA against the genes used in the present study. The expression of each gene within a sample was normalized against 36B4 mRNA expression and expression relative to the control sample using the formula 2^{-(ΔΔCt)} in which ΔΔCt=(Ct mRNA−Ct 36B4)ₛₐₘₚₗₑ−(Ct mRNA−Ct 36B4)_{control sample}.

Results

The data presented in FIG. 5 demonstrates that siRNA-loaded, peptide-conjugated glucan shells attenuate osteopontin (OPN) mRNA expression in adipose tissue of obese mice. Briefly, 5-week old ob/ob mice were injected once a day for 5 days with 1 mg of sDP (14)-GS loaded with 10 nmoles of Scr or OPN siRNA (FIG. 5a). On day 7, mice were sacrificed and epididymal adipose tissue (AT) was isolated. Total RNA was extracted and OPN, F4/80, and TNF-α gene expression was measured by real-time PCR (FIGS. 5b-d). Statistically significant knockdown was observed in adipose tissue of mice treated with OPN siRNA-loaded sDP (14)-GS (pcGP-14). Importantly, the expression of other macrophage and immune cell factors, including F4/80 and TNF-α, were unchanged in mice treated with pcGP-14s loaded with OPN siRNA compared with pcGP-14s loaded with Scr siRNA confirming the specificity of the siRNA-mediated knockdown (FIGS. 5c and 5d).

The exemplary OPN siRNA sequence is as follows:

(SEQ ID NO: 52)

5′-CCACAUGGCUGGUGCCUGA-3′

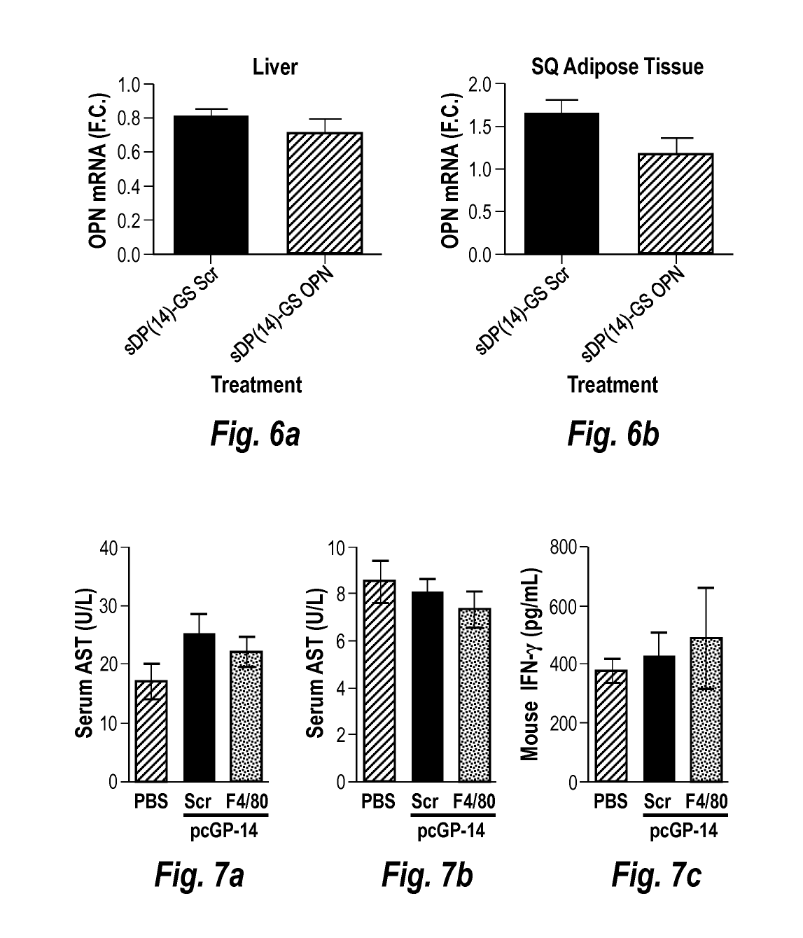

The sDP(14)-GS (pcGP-14) mediated gene silencing was shown to be specific to the epididymal adipose tissue. OPN expression was assessed in the liver versus subcutaneous (SQ) adipose tissue of mice treated with particles loaded with OPN siRNA versus scrambled control. FIG. 6 shows the expression of OPN in (a) liver and (b) subcutaneous (SQ) adipose tissue from mice treated for 5 days with sDP(14)-GS loaded with Scr (blue) or OPN (red) siRNA. n=10, statistical significance was determined by student's t-test. Results are mean±s.e.m.

The data presented in FIG. 5e-f demonstrates sDP (14)-GS (pcGP-14) mediated OPN silencing in adipose tissue improves glucose tolerance. Briefly, 5-week old ob/ob mice were injected once a day for 5 days with PBS or 1 mg sDP (14)-GS (pcGP-14) loaded with 10 nmoles of Scr or OPN siRNA. On day 6, mice were fasted overnight. On day 7, glucose tolerance tests (GTT) were performed. Briefly, blood glucose was measured before and 15, 30, 60, and 90 minutes after i.p. injecting 1 g/kg of glucose. Area under the curve (AUC) calculations were made and statistical significance was determined. Statistically significant improvement in glucose tolerance was observed in adipose tissue of mice treated with OPN siRNA-loaded sDP (14)-GS (pcGP-14). (FIG. 5e-f) There was no effect of pcGP-14s loaded with Scr siRNA on glucose tolerance. These data suggest that OPN silencing improves whole body glucose tolerance in ob/ob mice and provides a potentially useful therapeutic strategy for the treatment of insulin resistance and diabetes.

### Example 8

### In Vivo Particle Toxicity in Healthy Mice

Toxicity and Immune Response

C57BL6/J male mice (8 weeks old) were i.p. injected once a day for 5 days with siRNA formulated in pcGP-14s at a dose of 1.2 mg/kg siRNA (1 mg of pcGP-14s containing 10 nmoles of siRNA). Twenty-four hours after the last injection, mice were sacrificed and blood samples were collected by cardiac puncture. Serum was obtained by centrifuging at 5,000 rpm for 10 min.

ELISA Assay

Serum cytokine levels were determined using enzyme-linked immunosorbent assay (ELISA) kits. Tumor necrosis factor-α (TNF-α) and interferon-γ (IFN-γ) levels were measured using mouse ELISA kits (Pierce, Rockford, Ill.) as recommended by the manufacturer.

AST and ALT Measurement

To test for liver toxicity, the levels of aspartate aminotransferase (AST) and alanine aminotransferase (ALT) activity in serum were measured using commercial kits (Abnova, Taiwan) according to the manufacturer's instructions.

Statistical Analysis.

The statistical significance of the differences in the means of experimental groups was determined by Student's t-test using GraphPad Prism v 6.0c software. For all tests, p≦0.05 was considered significant. The data are presented as the means±SEM.

Results

Liver toxicity of pcGP-14s was assessed by measuring aspartate aminotransferase (AST) and alanine aminotransferase (ALT) activities in serum. No significant changes in either AST or ALT were detected in mice treated with pcGP-14s when compared to mice treated with PBS (FIGS. 7a and 7b). In addition, the production of pro-inflammatory cytokines, interferon (IFN)-γ and tumor necrosis factor (TNF)-α, was analyzed in the serum of particle-treated mice. pcGP-14 administration did not induce the production of IFN-γ (FIG. 7c). No TNF-α expression (<9 pg/mL) was detected following particle treatment.

### Example 9

### Discussion of Data and Results from Examples 1-8

This application describes the development of a simplified, single-component system to efficiently deliver therapeutic siRNA to phagocytic cells in vivo (FIGS. 3 and 5). This delivery system consists of glucan microparticles that have been functionalized with amphipathic peptides. Covalent attachment of a peptide to the glucan particle enables electrostatic binding of siRNA to this delivery system (FIGS. 2a and 2b). This is a significant improvement over the art-described a method for loading siRNA within glucan particles that consisted of three independent steps (FIG. 1a). The prior art loading strategy involved (1) forming siRNA complexes with the amphipathic peptide Endoporter (EP), (2) loading the EP/siRNA complexes in a hydrodynamic volume into the glucan particles, and finally (3) entrapping the complexes in the particles. In contrast, the new peptide-modified glucan particles can be loaded with siRNA in a single step simply by mixing siRNA with glucan particles previously conjugated with peptide (FIG. 1b).

The formulation that gave the highest siRNA binding capacity, and therefore the one chosen for further study, was the glucan particle modified with a 14-amino acid amphipathic peptide (pcGP-14). This peptide sequence consists of leucine and histidine residues, arranged in a particular structural conformation. In the context of the pcGP-14s, the peptide serves multiple functions, including directly binding the siRNA and facilitating the siRNA release from the endosome to the cytosol. It is hypothesized that the weak-base histidine residues of the peptide assist in the endosomal escape of the encapsulated siRNA by permeabilizing the endosomal membrane upon acidification within the endosome. This mechanism of endosomal escape is believed to be in common with that of other histidine-rich peptides.

The new pcGPs combine straightforward synthesis and siRNA loading with the additional benefit of phagocyte-specificity conferred by the glucan particle. The glucan particle employed in the pcGPs is composed primarily of β-1,3-D-glucan and previous studies showed that these particles can deliver cargo specifically to phagocytic cells, such as macrophages and dendritic cells. This key feature of the glucan particles is particularly attractive for RNAi therapeutic applications where delivery of siRNA to specific cell types is desired. Selectively targeting phagocytic cells, such as macrophages, should reduce the adverse side effects associated with non-specific silencing of genes throughout the body. In initial experiments, it was demonstrated that significant silencing of F4/80 gene expression could be achieved in peritoneal macrophages following i.p. injection of pcGP-14s loaded with an F4/80-targeting siRNA (FIG. 3c). Treatment of healthy mice with GeRP-14s did not induce liver toxicity or the production of inflammatory cytokines (FIG. 3).

Phagocytic cells, such as macrophages, represent potentially important targets for RNAi therapeutics on the basis of their role in mediating inflammation and immune responses. Macrophages play an important role in the pathogenesis of many inflammatory diseases, including psoriasis, asthma, rheumatoid arthritis, and inflammatory bowel disease. In addition, macrophages contribute to the progression of neurodegeneration, atherosclerosis, fibrosis, cancer, and diabetes. Therefore, development of technology that can deliver siRNA to macrophages would offer a significant advance in the treatment of these and other major human diseases. A number of recent studies have described the delivery of siRNA to phagocytic cells for the treatment of inflammatory diseases. Despite this progress, several major challenges to the clinical translation of siRNA as therapy remain.

It is demonstrated herein the utility of the pcGPs for the treatment of inflammation-related diseases by reducing inflammatory cytokine expression in a mouse model of obesity and insulin resistance. Obesity rates in the US and worldwide are increasing at an alarming rate, and have resulted in a rise in related health problems such as diabetes and cardiovascular disease. Several laboratories (including that of the instant inventors) have shown that macrophages accumulate in the adipose tissue of obese rodents and humans, where they secrete a variety of inflammatory cytokines, such as TNF-α and OPN, that can exacerbate insulin resistance. Previous studies have reported that gene ablation of chemokines or blocking cytokines by injection of antibodies can, in some cases, alleviate insulin resistance. Importantly, reducing expression of these pro-inflammatory cytokines using the pcGP technology may result in an improved whole-body glucose tolerance. Thus, adipose tissue inflammation may represent a promising target for siRNA-based therapy in patients with obesity and metabolic disease.

Using the pcGPs of the invention, the instant inventors have demonstrated a significant 80% reduction of OPN expression in the visceral epididymal AT of ob/ob mice following i.p. administration of pcGP-14s loaded with OPN siRNA compared to pcGP-14s loaded with a control Scr siRNA (FIG. 5b). Importantly, the pcGP-14s silence genes selectively in phagocytic cells in the inflamed epididymal AT, with no silencing observed in other tissues such as the subcutaneous AT or liver. Thus, the pcGP-14s reduce inflammation only in the inflamed tissue while leaving other tissues unaffected. Silencing OPN in macrophages of the epididymal AT with the pcGP-14s resulted in an improved metabolic phenotype (FIGS. 5e and 5f). The pcGP-14s are simple to prepare and easily loaded with siRNA, both attractive qualities in terms of clinical application of a delivery vehicle. In addition, they are a versatile system that can potentially be used to deliver siRNA targeting any gene of interest. Due to their ability to provide efficient siRNA delivery to phagocytic cells with minimal toxicity, we envision the pcGP-14s being utilized for the treatment of a variety of inflammation-related diseases.

### CONCLUSIONS

In summary, peptide-conjugated GPs are a simplified single-component system that can efficiently deliver siRNA to phagocytic cells in vivo. Peptide-conjugated GPs were synthesized by covalently conjugating amphipathic peptides to the GPs. Peptide modification of the GPs facilitated the binding of siRNA to the GPs by electrostatic interaction. The new peptide-conjugated GPs can be loaded with siRNA simply by mixing siRNA with the modified GPs. In vivo evaluation demonstrated that the pcGP-14s can efficiently deliver siRNA and reduce F4/80 gene expression in macrophages of lean, healthy mice with minimal toxicity. Additionally, pcGP-14s can mediate gene silencing of OPN (an inflammatory cytokine) in the inflamed adipose tissue in obese mice. Importantly, reducing the expression of OPN in adipose tissue macrophages of obese mice improved the glucose tolerance. Thus, peptide-conjugated GPs, and specifically pcGP-14s, represents a promising new system for the efficient delivery of siRNA to phagocytic cells in vivo. It is believed that the potential of this technology in the treatment of other inflammation-related diseases is significant.

### Example 10

### Design and Testing of Further pcGPs and/or amGPs of the Invention

Further pcGPs and/or amGPs were designed with the aim of protect and retain the siRNA in the acidic environment of the inflamed gut and release siRNA in the cytoplasm upon phagocytosis for the application in IBD.

The pcGPs and amGPs in Table 4 were prepared according to the protocol outlines in FIGS. 1 b-c, as described above. Small molecule amines or amphipathic peptides (having amide modified C-terminal ends) were selected based on their predicted ability to bind siRNA and facilitate subsequent release into the cytoplasm.

TABLE 4
Table 4: Representative Modifications on Glucan Shell

| | |
|---|---|
| small molecule or short peptide | mol:mol ratio |

Ethylenediamine

| | |
|---|---|
| Ethylenediamine:Histamine | 75:25 |
| Ethylenediamine:Histamine | 50:50 |
| Ethylenediamine:Histamine | 25:75 |

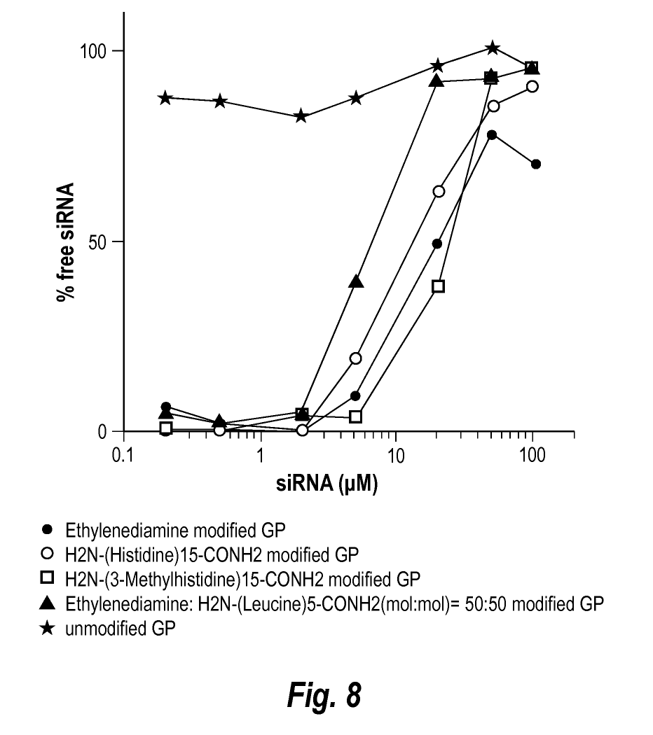

Histamine
H₂N-(Histidine)₁₅-CONH₂
H₂N-(3-Methylhistidine)₁₅-CONH₂

| | |
|---|---|
| Ethylenediamine:H₂N-(Leucine)₅-CONH₂ | 50:50 |
| Ethylenediamine:H₂N-(Leucine)₁₅-CONH₂ | 50:50 |
| H₂N-(Histidine)₁₅-CONH₂:H₂N-(Leucine)₅-CONH₂ | 50:50 |
| H₂N-(Histidine)₁₅-CONH₂:H₂N-(Leucine)₁₅-CONH₂ | 50:50 |

Where indicated, GPs were modified using mixtures of small molecules amines, mixtures of amphipathic (delivery) peptides, or mixtures of small molecule amines and amphipathic (delivery) peptides. Molar ratios are indicated, where appropriate. Certain exemplary pcGPs or amGPs were tested for their ability to efficiently bind siRNA by determining the percentage of free siRNAs after the formation of particle:siRNA electrostatic complexes. Data are set forth in FIG. 8.

pcGPs or amGPs (1 mg/ml) were incubated with different concentrations of fluorescent siRNAs to form electrostatic complexes in sodium acetate buffer (pH=4.8). After spinning down at 5000 rpm for 5 min, siRNA/GeRP complexes were precipitated, while free siRNAs were remained in solution. Percentage of free siRNA was then measured and plotted against siRNA concentration to determine the binding affinity of particles.

### Example 11

### Macrophage Uptake of amGPs

Several candidate amGPs were identified which are efficiently internalized by PECs in vitro. Fluorescent microscopy showing an example of new FITC-amGP formulations (green), ethylenediamine modified GP, internalized by PECs (red) in vitro. FITC-labeled new amGPs (10 μg/ml) were incubated with PECs for 1 h. PECs were then washed with PBS and fixed by 4% formaldehyde. Fixed cells were washed again with 0.3% PBST and incubated in blocking buffer (5% normal goat serum) for 30 min at room temperature. Cells were then incubated with primary antibody against F4/80 at 4′C overnight. After that, secondary antibody with AlexaFluor647 dye (red) was added and incubated for 30 min at room temperature. Cells were finally washed with PBS and mounted with Prolong Gold with DAPI. Nuclei were stained with DAPI (blue). Green fluorescence co-localized with F4/80 marker fluorescence indicating updake/phagocytosis of amGPs by macrophages (PECs) (data not shown.)

### Example 10

### Test New amGP Formulations for Efficient TNF-α Silencing in IBD Models In Vivo and their Therapeutic Potential for the Treatment of IBD

Select amGP formulations validated above were tested in IBD models for their ability to silence TNF-α expression and decrease IBD symptoms as well as their therapeutic potential for the treatment of IBD.

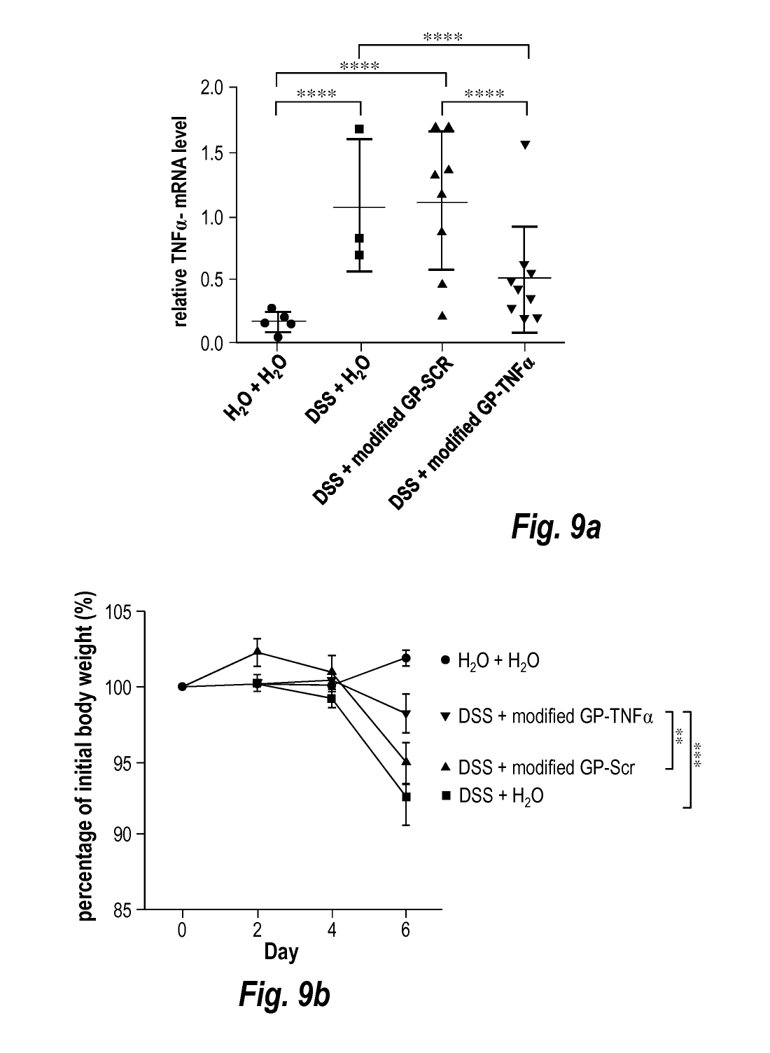

Preliminary data indicated that one of the new amGP formulations, ethylenediamine modified GPs, significantly decreased TNF-α expression in distal colons by oral delivery in DSS-induced colitis mouse model, without affecting circulating TNF-α levels in serum. In addition, silencing with this new formulation reduced the weight loss associated with colitis (FIG. 9). FIG. 9a depicts silencing of TNF-α expression in distal colons and FIG. 9b depicts reduction in body weight loss of DSS-treated mice, by oral treatment of ethylenediamine-modified GP formulation in DSS-induced colitis mouse model. C57BL6 male mice treated with water or 3% DSS for 7 days were given water, new amGPs with control scrambled (SCR) siRNA or with siRNA targeting TNF-α by oral gavage on days 2, 4, and 6. Weight loss was monitored during the treatment. On day 7, different parts of the colon were harvested and RNA was prepared by RNeasy Plus Mini Kit (Qiagen). TNF-α mRNA level was determined by quantitative RT-PCR. (**p≦0.01,***p≦0.001,****p≦0.0001) The exemplary TNF-α sequence is as follows:

(SEQ ID NO: 53)

5′-GUGAAUGAGUGUCAAGAUA-3′.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein can be utilized according to such related embodiments of the present invention. Thus, the following claims are intended to encompass within their scope modifications, substitutions, and variations to processes, manufactures, compositions of matter, compounds, means, methods, and/or steps disclosed herein.

The contents of any patents, patent applications, and references cited throughout the specification are herein incorporated by reference in their entireties.

We claim:

1. A preparation of peptide-conjugated (pc) yeast cell wall particles (YCWPs), wherein the preparation comprises peptide, e.g., delivery peptide, conjugated to components, e.g., oligosaccharides, within the cell wall of the YCWPs.

2. A peptide-conjugated (pc) yeast cell wall particle (YCWP) delivery system, comprising (YCWPs) comprising yeast cell wall components, e.g., oligosaccharides, conjugated to peptides, e.g., delivery peptides, wherein the system comprises a nucleic acid payload molecule complexed with said peptide.

3. The preparation or system of claim 1 or 2, wherein the peptide is selected from the group consisting of a cationic peptide, an amphipathic peptide, and a polyhistidine peptide.

4. The preparation or system of claim 3, wherein the peptide is an amphipathic peptide.

5. The preparation or system of claim 4, wherein the peptide has a length of about 5 to about 30 residues.

6. The preparation or system of claim 4 or 5, wherein the peptide consists of alternating lipophillic and basic residues, optionally in pairs, optionally alternating with single lipophillic or basic residues.

7. The preparation or system of claim 6, wherein the lipophillic residue is leucine (L) and the basic residue is histidine (H) or lysine (K).

8. The preparation or system of claim 6, wherein the peptide has about 50% lipophillic and 50% basic or weakly basic residues.

9. The preparation or system of claim 6, wherein the peptide has about 40% lipophillic and 60% basic residues.

10. The preparation or system of claim 6, wherein the peptide has about 60% lipophillic and 40% basic residues.

11. The preparation or system of claim 4 or 5, wherein the peptide has the formula [(A)ₙ₌₁₋₂ (B)ₙ₌₁₋₂]ₙ₌₅₋₃₀.

12. The preparation or system of claim 4, wherein the peptide is selected from the group consisting of:
(SEQ ID NO: 1)
H₂N-LHHLLHHLLHHLHHLLHHLHHLLHHL-COOH,
(SEQ ID NO: 3)
H₂N-LHKLLHHLLHHLHKLLHHLHHLLHKL-COOH
(SEQ ID NO: 2)
H₂N-LHKLLHHLLHKLHHLLHKLHHLLHHL-COOH
(SEQ ID NO: 4)
H₂N-LHHLLHHLLHHLHHL-COOH
(SEQ ID NO: 5)
H₂N-HHLLHHLHHLLHHL-COOH
(SEQ ID NO: 6)
H₂N-LHLLHHLLHHLHHL-COOH,
(SEQ ID NO: 7)
H₂N-LHHLLHLLHHLLHHL-COOH,
(SEQ ID NO: 8)
H₂N-LHKLLHHLLHHLHK-COOH
(SEQ ID NO: 9)
H₂N-LHKLLHHLHHLLHKL-COOH
(SEQ ID NO: 10)
H₂N-KLHHLLHKLHHLLHH-COOH
(SEQ ID NO: 11)
H₂N-HLHLLHHLLHH-COOH,
(SEQ ID NO: 12)
H₂N-LHLLHHLLHH-COOH,
(SEQ ID NO: 13)
H₂N-LHKLLHHLLHKLHHL-COOH
(SEQ ID NO: 14)
H₂N-LHLLHH-COOH,
(SEQ ID NO: 15)
H₂N-LHHLL-COOH,
and
(SEQ ID NO: 16)
H₂N-LHKLL-COOH.

13. The preparation or system of claim 4, wherein the peptide is selected from the group consisting of:
(SEQ ID NO: 34)
H₂N-LHHLLHHLLHHLHHLLHHLHHLLHHL-CONH₂,
(SEQ ID NO: 35)
H₂N-LHKLLHHLLHHLHKLLHHLHHLLHKL-CONH₂
(SEQ ID NO: 36)
H₂N-LHKLLHHLLHKLHHLLHKLHHLLHHL-CONH₂
(SEQ ID NO: 37)
H₂N-LHHLLHHLLHHLHHL-CONH₂
(SEQ ID NO: 38)
H₂N-HHLLHHLHHLLHHL-CONH₂
(SEQ ID NO: 39)
H₂N-LHLLHHLLHHLHHL-CONH₂,
(SEQ ID NO: 40)
H₂N-LHHLLHLLHHLLHHL-CONH₂,
(SEQ ID NO: 41)
H₂N-LHKLLHHLLHHLHK-CONH₂
(SEQ ID NO: 43)
H₂N-LHKLLHHLHHLLHKL-CONH₂
(SEQ ID NO: 43)
H₂N-KLHHLLHKLHHLLHH-CONH₂
(SEQ ID NO: 44)
H₂N-HLHLLHHLLHH-CONH₂,
(SEQ ID NO: 45)
H₂N-LHLLHHLLHH-CONH₂,
(SEQ ID NO: 46)
H₂N-LHKLLHHLLHKLHHL-CONH₂
(SEQ ID NO: 47)
H₂N-LHLLHH-CONH₂,
(SEQ ID NO: 48)
H₂N-LHHLL-CONH₂,
and
(SEQ ID NO: 49)
H₂N-LHKLL-CONH₂.

14. The preparation or system of claim 3, wherein the peptide is a polyhistidine peptide.

15. The preparation or system of claim 14, wherein the peptide comprises 2-20, 2-16, 2-10, 2-8 or 2-6 histidines.

16. The preparation or system of claim 14, wherein the peptide is selected from the group consisting of:
(SEQ ID NO: 17)
H₂N-HH-COOH,
(SEQ ID NO: 18)
H₂N-HHHHHH-COOH,
(SEQ ID NO: 19)
H₂N-HHHHHHHH-COOH,
(SEQ ID NO: 20)
H₂N-HHHHHHHHHHHHHHH-COOH,
(SEQ ID NO: 54)
H₂N-HH-CONH₂,
(SEQ ID NO: 55)
H₂N-HHHHHH-CONH₂,
(SEQ ID NO: 56)
H₂N-HHHHHHHH-CONH₂,
(SEQ ID NO: 57)
H₂N-HHHHHHHHHHHHHHH-CONH₂.

17. The preparation or system of claim 3, wherein the peptide is a cationic peptide.

18. The preparation or system of claim 17, wherein the cationic peptide a polyarginine peptides, a cell-penetrating peptides or other synthetic cationic peptide.

19. The preparation or system of claim 18, wherein the peptide is selected from the group consisting of:
(SEQ ID NO: 25)
Penetratin - RQIKIWFQNRRMKWKK
(SEQ ID NO: 26)
Transportan - LIKKALAALAKLNIKGLLYGASNLTWG
(SEQ ID NO: 27)
EB1 - LIRLWSHLIHIWFQNRRLKWKKK
(SEQ ID NO: 28)
TAT - GRKKRRQRRRPPQ
(SEQ ID NO: 29)
MPG - GALFLGFLGAAGSTMGAWSQPKKKRKV
(SEQ ID NO: 30)
CADY - GLWRALWRLLRSLWRLLWRA
(SEQ ID NO: 31)
MAP - KLALKLALKALKAALKLA
(SEQ ID NO: 32)
Polyarginine - RRRRRRRRR
(SEQ ID NO: 33)
bPrPp - MVKSKIGSWILVLFVAMWSDVGLCKKRPKP

20. The preparation or system of claim 3, wherein the peptide is a polyleucine peptide.

21. The preparation or system of claim 20, wherein the peptide is selected from the group consisting of:
(SEQ ID NO: 21)
H₂N-LL-COOH,
(SEQ ID NO: 22)
H₂N-LLLLL-COOH,
(SEQ ID NO: 23)
H₂N-LLLLLLLLLL-COOH,
(SEQ ID NO: 24)
H₂N-LLLLLLLLLLLLLLL-COOH,
(SEQ ID NO: 58)
H₂N-LL-CONH₂,
(SEQ ID NO: 59)
H₂N-LLLLL-CONH₂,
(SEQ ID NO: 60)
H₂N-LLLLLLLLLL-CONH₂
(SEQ ID NO: 61)
H₂N-LLLLLLLLLLLLLLL-CONH₂.

22. A preparation of amine-modified (am) yeast cell wall particles (YCWPs), wherein the preparation comprises an amine, e.g., a small molecule amine, conjugated to components, e.g., oligosaccharides, within the cell wall of the YCWPs.

23. An amine-modified (am) yeast cell wall particle (YCWP) delivery system, comprising (YCWPs) comprising yeast cell wall components, e.g., oligosaccharides, conjugated to amines, e.g., small molecule amines, wherein the system comprises a nucleic acid payload molecule complexed with said peptide.

24. The preparation or system of claim 22 or 23, wherein the small molecule amine is selected from the group consisting of

[symbol]

25. The preparation or system of claim 22 or 23, wherein the small molecule amine is a primary, secondary or tertiary amine.

26. The preparation or system of claim 22 or 23, wherein the small molecule amine is selected from those set forth in Table 2.

27. The preparation or system of any one of the preceding claims, wherein the peptide or amine is conjugated to a moiety in the yeast cell wall particle via a linker moiety.

28. The preparation or system of claim 27, wherein the linker moiety is a non-degradable linker moiety.

29. The preparation or system of claim 28, wherein the linker moiety is selected from the group consisting of an amine linkage, an amide linkage, a carbonate linkage, a carbamate linkage, an ether linkage, a thioether linkage, an oxime linkage and a triazole linkage.

30. The preparation or system of claim 27, wherein the linker moiety is a degradable linker moiety.

31. The preparation or system of claim 30, wherein the linker moiety is selected from the group consisting of a hydrazone linkage, an acetal linkage, a ketal linkage, a thioketal linkage, a disulfide linkage, an ester linkage, an orthoester linkage and an anhydride linkage.

32. The preparation or system of any one of the preceding claims, further comprising a nucleic acid payload.

33. The preparation or system of claim 32, wherein the nucleic acid payload is a siRNA.

34. A method of effecting gene silencing, e.g., RNAi, of a target gene or mRNA of a target gene in a cell, the method comprising contacting the cell with an effective amount of the preparation or delivery system of claim 32 or 33, and incubating said cell under conditions such that the target gene is effectively silenced.

35. The method of claim 34, wherein the cell is contacted in vitro.

36. The method of claim 34, wherein the cell is contacted in vivo.

37. A method of effecting gene silencing, e.g., RNAi, of a target gene or mRNA of a target gene in an organism, the method comprising administering to the organism an effective amount of the preparation or delivery system of claim 32 or 33, under conditions such that the target gene is effectively silenced.

38. The method of claim 37, wherein the subject has, or is at risk for, developing a disease or disorder associated with the presence of the target gene or mRNA of said gene.

39. A kit comprising the preparation or delivery system of the preceding claims, further including instructions for use.

40. A method of making the preparation of claim 1, comprising contacting a preparation of YCWPs with the peptide, wherein the peptide is modified with a linker moiety, under conditions such that the peptide is conjugated to a component of the YCWP via said linker moiety.

41. A method of making the delivery system of claim 2, comprising contacting a peptide-conjugated (pc) YCWP with a nucleic acid payload molecule under conditions such that the nucleic acid payload molecule complexes with the peptide within the pcYCWP.

42. A method of making the preparation of claim 22, comprising contacting a preparation of YCWPs with the amine, wherein the amine is modified with a linker moiety, under conditions such that the amine is conjugated to a component of the YCWP via said linker moiety.

43. A method of making the delivery system of claim 23, comprising contacting an amine-modified (am) YCWP with a nucleic acid payload molecule under conditions such that the nucleic acid payload molecule complexes with the amine within the pcYCWP.

44. The method of any one of the preceding claims, wherein the nucleic acid payload molecule is a siRNA.
